(19) United States
(12) Patent Application Publication
(10) Pub. No.: US 2022/0160991 A1
(43) Pub. Date: May 26, 2022

(54) SYSTEM AND METHOD FOR DATA COLLECTION, RESEARCH, AND PROACTIVE MEDICAL TREATMENT

(71) Applicant: 8B MEDICAL LLC, Los Altos, CA (US)

(72) Inventors: Meghan Barrier Craig, Cle Elum, WA (US); Scott Andrew Forman, Albuquerque, NM (US); Dean Alan Hovey, Los Altos, CA (US); Chris Augustine Luomanen, San Francisco, CA (US); Douglas Andrew Romney, Fairhaven, MA (US); Cameron András Tacklind, Menlo Park, CA (US); Christopher Andrew Tacklind, Menlo Park, CA (US); Eric Robert Vicenti, Santa Monica, CA (US)

(73) Assignee: 8B MEDICAL LLC, Los Altos, CA (US)

(21) Appl. No.: 17/533,060

(22) Filed: Nov. 22, 2021

Related U.S. Application Data
(60) Provisional application No. 63/117,203, filed on Nov. 23, 2020.

(51) Int. Cl. A61M 16/20

(52) U.S. Cl. CPC A61M 16/202; A61M 2016/0027; A61M 16/208

(57) ABSTRACT

A diagnostic tool can include a face mask, a casing, a plurality of sensors, and processing circuitry. The face mask can include an air-intake port, a first check valve integrated into the air-intake port, an air-exhaust port, and a second check valve integrated into the air-exhaust port. The casing can be coupled to the face mask having an air-intake chamber coupled to the air-intake port and an air-exhaust chamber coupled to the air-exhaust port. The processing circuitry can be communicatively coupled to the plurality of sensors. The processing circuitry can include computing logic for handling information detected by the plurality of sensors.

### TECHNICAL FIELD

The present invention relates to systems and methods for a medical device that can automate therapeutic and diagnostic techniques and perform research to improve such techniques.

### CROSS-REFERENCE TO RELATED APPLICATIONS

This application claims priority under 35 U.S.C. 119(e) to U.S. Provisional Application No. 63/117,203 filed Nov. 23, 2020.

### BACKGROUND

Medical devices are used to facilitate patient respiration by providing a positive source of air flow and regulating air pressures. Typical respiratory medical devices rely on mechanical pressure-regulating valves, manual observation of patient conditions, and manual adjustment of operations of a device for therapeutic and diagnostic purposes. Such mechanisms are costly, require attention from care providers, and cannot address nuanced patient conditions.

Therefore, there exists a need in the art for improved respiratory devices that can automatically adjust operation in response to detected patient conditions and improved research and diagnostic techniques based on such observations.

### SUMMARY

The present invention is generally directed to systems and methods for a medical device that can automate therapeutic and diagnostic techniques and perform research to improve such techniques. A system executing the methods can be directed by a program stored on non-transitory computer-readable media.

An aspect can include a patient interface manifold. The manifold can be used for modulating air-flow rate. The patient interface manifold can include a casing, a first peripheral air-flow port, a second peripheral air-flow port, a patient air-flow port, a valve, and processing circuitry. A casing can include a plurality of chambers. The position of a valve can regulate air-flow rate in a plurality chambers of a casing. Processing circuitry can include computing logic to control a valve to modulate the air-flow rate in a plurality of chambers. A first peripheral air-flow port can be connected to a first chamber of the casing. A second peripheral air-flow port can be connected to a second chamber of the casing. A patient air-flow port can be connected to a third chamber of the casing.

In an embodiment, a valve can be a rotary disc valve having a first rotary disc and a second rotary disc. A first rotary disc can include, for example, two apertures. The first rotary disc can be substantially fixed to align the apertures of a first rotary disc with chambers of a casing. A second rotary disc can include, for example, an aperture. A second rotary disc can be configured to rotate.

In another embodiment, apertures of a second rotary disc have a concave curved edge.

In yet another embodiment, a patient interface manifold can include a plurality of air-flow port bodies. Such bodies can be communicatively coupled to processing circuitry. In another sense, such bodies can be communicatively coupled to one or more of a first air-flow port body disposed in a first chamber and/or a second air-flow port body disposed in a second chamber. Air-flow port bodies can be configured to detect air-flow rate. Air-flow port bodies can be configured to communicate information about a detected air-flow rate to processing circuitry. In some embodiments, a plurality of air-flow port bodies can have a pitot tube geometry.

Another embodiment can include a sensor. A sensor can be communicatively coupled to processing circuitry. A sensor can be arranged and/or configured to detects various conditions, such as temperature, humidity, and/or gas content of the first chamber.

In yet another embodiment, processing circuitry can transmit information to a patient computer.

Another aspect can include a diagnostic tool. A diagnostic tool can include a face mask, a casing, one or more sensors, and processing circuitry. A face mask can include an air-intake port, a first check valve integrated into the air-intake port, an air-exhaust port, and/or a second check valve integrated into the air-exhaust port. A casing can be coupled to a face mask. The face mask can include an air-intake chamber. The air-intake chamber can be coupled to an air-intake port. The face mask can include an air-exhaust chamber. The air-exhaust chamber can be coupled to an air-exhaust port. Processing circuitry can be communicatively coupled to a sensor and/or a plurality of sensors. Processing circuitry can include computing logic. Computing logic can be configured to handle information detected by a sensor and/or a plurality of sensors.

In an embodiment, computing logic can be capable of directing storage of information. The information can include information detected by a sensor and/or a plurality of sensors.

In another embodiment, a sensor can be a two-electrode echocardiogram sensor, a skin-temperature sensor, blood-oxygen sensor, a pulse sensor, a blood-pressure sensor, or a gas-content sensor. A plurality of sensors can include any combination of the foregoing sensors.

In an embodiment, a plurality of sensors can includes a first gas-content sensor and a second gas-content sensor. The first gas-content sensor can be disposed in an air-intake chamber. The second gas-content sensor can be disposed in the air-exhaust chamber.

In another embodiment, a plurality of sensors can include a first air-pressure sensor and a second air-pressure sensor. The first air-pressure sensor can be disposed in an air-intake chamber. The second air-pressure sensor can be disposed in an air-exhaust chamber.

Yet another aspect can include a powered air filter. A powered air filter can include a casing, an air blower, processing circuitry, and a power source. A casing can include an air-flow port. An air-flow port can be couplable to a face mask. An air blower can be contained within a casing. Processing circuitry can be coupled to an air blower. Processing circuitry can include computing logic. The computing logic can be configured to modulate the speed of an air blower.

In an embodiment, a powered air filter can include an air-pressure sensor. An air-pressure sensor can be contained within a casing. An air-pressure sensor communicatively coupled to processing circuitry. An air-pressure sensor can be configured to detect an air pressure between an air blower and an air-flow port. Processing circuitry can be capable of modulating the speed of an air blower based on, for example, information detected by an air-pressure sensor.

In another embodiment, processing circuitry can be capable of modulating the speed of an air blower based on, for example, a state-space model. A state-space model can include the speed and/or velocity of the air blower.

In yet another embodiment, a casing can be constructed from air-filter material.

### BRIEF DESCRIPTION OF THE DRAWINGS

The present invention is further described in the detailed description which follows, in reference to the noted plurality of drawings by way of non-limiting examples of certain embodiments of the present invention, in which like numerals represent like elements throughout the several views of the drawings, and wherein:

FIG. 1 depicts an oblique exterior view of a patient interface manifold with a spring-based pressure-limiting valve.

FIG. 2 depicts a cross-section view of a patient interface manifold with a spring-based pressure-limiting valve and an obstruction-based air-flow sensor.

FIG. 3 depicts an underside view of a patient interface manifold with an air-pressure sensor and a Universal Serial Bus peripheral cable.

FIG. 4 depicts an oblique exterior view of a patient interface manifold with a single aperture rotary disc valve.

FIG. 5 depicts an oblique cross-section view of a patient interface manifold with a single aperture rotary disc valve and an obstruction-based air-pressure sensor.

FIG. 6 depicts a cross-section view of a patient interface manifold with a single aperture rotary disc valve and an obstruction-based air-pressure sensor.

FIG. 7 depicts an underside exterior view of a patient interface manifold with a single aperture rotary disc valve.

FIG. 8 depicts an oblique exterior view of a patient interface manifold with a dual aperture rotary disc valve and an obstruction-based air-pressure sensor.

FIG. 9 depicts an oblique horizontal cross-section view of a patient interface manifold with a dual aperture rotary disc valve.

FIG. 10 depicts a dual aperture rotary disc valve in situs viewed from the patient side.

FIG. 11 depicts a cross-section view of a patient interface manifold with a dual aperture rotary disc valve, including installed flow sensors.

FIG. 12 depicts an oblique exterior view of a patient interface manifold with a dual aperture rotary disc valve with space for inserting removable bilateral air pressure sensors, without an enclosing case.

FIG. 13 depicts an oblique exterior view of a patient interface manifold with a dual aperture rotary disc valve without a circuit board or casing, including ports for sensors.

FIG. 14 depicts a cross section view of a patient interface manifold with symmetrical inhalation and exhalation apertures with air-flow sensing port bodies inserted into the airways.

FIG. 15 depicts a cross section of a patient interface manifold including the circuit board and casing.

FIG. 16 depicts an oblique view of a patient interface manifold including the full-body casing.

FIG. 17 depicts an oblique view of an obstruction-based air-flow port body.

FIG. 18 depicts a cross section of an obstruction-based air-flow port body.

FIG. 19 depicts an oblique view of a pitot-tube-based air-flow port body.

FIG. 20 depicts a cross section view of a pitot-tube-based air-flow port body.

FIG. 21 depicts a configuration of a ventilator circuit using an air blower with minimal tubing during an inhalation state.

FIG. 22 depicts a configuration of a ventilator circuit using an air blower with minimal tubing during an exhalation state.

FIG. 23 depicts a block diagram of a medical device connected to a patient computer.

FIG. 24 depicts network topologies for medical devices, patient computers, on-site remote computing systems, and off-site remote computing systems.

FIG. 25 depicts a user interface for configuring a ventilator mode.

FIG. 26 depicts a user interface for changing an inspiratory:expiratory ratio operating setting.

FIG. 27 depicts a user interface for changing an operating setting, namely a positive-end expiratory pressure parameter.

FIG. 28 depicts a user interface for setting a target fraction of inspired oxygen for a device that lacks direct oxygen control.

FIG. 29 depicts an advanced menu of a user interface for a ventilator device.

FIG. 30 depicts a user interface for shutting down a ventilator device.

FIG. 31 depicts a user interface for configuring a pressure support mode.

FIG. 32 depicts a user interface for configuring alarms for a medical device.

FIG. 33 depicts a direct-to-patient air blower device.

FIG. 34 depicts an underside view of a direct-to-patient air blower device with the bottom casing removed.

FIG. 35 depicts an underside view of a direct-to-patient air blower device including the bottom casing.

FIG. 36 depicts a vertical cross-section view of a direct-to-patient air blower device, including a circuit board.

FIG. 37 depicts a rear view of a compact ventilator device.

FIG. 38 depicts a compact ventilator without a rear panel.

FIG. 39 depicts an oblique cross-section of a bag squeezer with a constraining mechanism in an open position.

FIG. 40 depicts a cross section of a bag squeezer with a constraining mechanism in a closed position.

FIG. 41 depicts a bag-squeezer with a constraining mechanism in an open position.

FIG. 42 depicts a bag squeezer in an open position.

FIG. 43 depicts a fixed disc with symmetrical sector-shaped apertures and a corresponding moving disc with an aperture with concave edges for a rotary disc valve.

FIG. 44 depicts a user interface for setting a tidal volume based on an ideal body weight.

FIG. 45 depicts an oblique exterior external-facing view of a powered air filter.

FIG. 46 depicts an oblique exterior patient-facing view of a powered air filter.

FIG. 47 depicts an exploded view of a powered air filter.

FIG. 48 depicts a cross-section of a powered air filter.

FIG. 49 depicts an oblique external-facing view of a diagnostic tool integrated into a mask with a sensor array.

FIG. 50 depicts an oblique exterior patient-facing of a diagnostic tool integrated into a mask with a sensor array.

### DETAILED DESCRIPTION

A detailed explanation of the system, method, and exemplary embodiments of the present invention are described below. Exemplary embodiments described, shown, and/or disclosed herein are not intended to limit the claims, but rather, are intended to instruct one of ordinary skill in the art as to various aspects of the invention. Other embodiments can be practiced and/or implemented without departing from the scope and spirit of the claimed invention.

Respiratory interfaces have been used to, e.g., provide patients with regulated air to facilitate breathing via a ventilator. Yet such interfaces have not been fully utilized. Embodiments described herein advance the utility of interfaces in several respects. For example, a patient interface manifold can provide centralized operating mechanisms for a medical device. A patient interface manifold can be a locus for data collection about operation of a medical device and patient conditions. A patient interface manifold can be a hub for data collection for use in research and diagnostics. A patient interface manifold can be a hub for implementing proactive therapies.

A positive air-flow device, such as a ventilator, can be connected to a recipient of positive air flow via a patient interface manifold. A patient interface manifold can include multiple ports to airways. For example, a first port can be a port to an airway from a positive air-flow device. A second port can be a port to an exhaust airway. A third port can be a port to an airway connected to an endotracheal tube.

A patient interface manifold can include several valves. For example, a valve of a patient interface manifold can be utilized to regulate air pressure in a cavity connected to a patient interface manifold. For example, the cavity can be a patient's lungs. A port of a patient interface manifold can connect to a patient's lungs via an endotracheal tube. Alternatively, the cavity can be a chamber of a patient interface manifold. A cavity can include the patient's lungs and the chamber of the patient interface manifold, as well as the volume of connecting hoses, etc. Operation of a valve can be automated.

A patient interface manifold can include sensors. For example, a sensor of a patient interface manifold can detect air pressure in a cavity. The sensor can transmit signals indicating, e.g., a detected air pressure of a cavity. Other types and utilizations of various sensors are contemplated and discussed herein.

A patient interface manifold can have ports including a patient port, a positive air-flow source port, and an exhaust port.

In an embodiment, a patient interface manifold can include a pressure-regulating valve. The pressure-regulating valve can be a pressure-limiting valve.

A pressure-limiting valve can include components such as a spring and a piston. The piston can be placed in a closed position, to prevent or limit air from escaping from a cavity, or an open position, to allow or increase the amount of air escaping from the cavity. The piston can be persuaded into position by a spring.

The spring can be compressed to exert a force on, e.g., a plug, or a piston. The force can be calibrated to maintain a first position yet allow other positions as desired. For example, a piston can move from a first position if an air pressure exerted on a piston's exposure to a cavity or chamber exceeds the spring's compression force. A piston can be utilized to open the valve, thereby compressing the spring, to reduce air pressure in a cavity. By moving the valve into an open position, air can escape from the cavity, thereby reducing air pressure in the cavity. A piston can be arranged to return to a first (closed) position, for example when the air pressure in a cavity (as measured by the pressure exerted on the area of the piston's exposure) reduces to less than or equal to the spring's bias.

A check valve can prevent air from flowing in an undesired direction, such as toward an air-flow source during exhalation.

A spring's bias can be modulated. For example, the spring can be compressed, thereby increasing force applied by the spring. Similarly, the spring compression value can be reduced by extending the compressed spring to a length nearer the length of the spring in its relaxed state. A desired air pressure limit or range can be achieved by, e.g., regulating the spring compression value to a desired air pressure limit or range.

Increasing and decreasing, or modulating, the spring's bias can be achieved via an actuator. The actuator can include a compression arm. An actuator can include a servomechanism to indicate a position of a compression arm or the spring's compression value. The actuator can utilize a motor, for example, a servomotor. An actuator can advantageously compress a spring to a precise, controllable compression value. Thus, air pressures can be precisely maintained. An actuator can compress a spring to set a precise compression pressure in response to a command, such as a user input and/or based on sensor feedback.

A patient interface manifold can utilize controllers, processors, and/or computing devices, which can transmit signals indicating commands to set precise pressures. For example, a controller, processor, or computing device can transmit a signal in order to set a desired air-pressure limit. The controller or computing device can transmit multiple signals to change the desired air pressure, for example in response to a user input, an automated change, and/or based on a change in patient condition detected by one or more sensors, such as taking a breath.

A first desired air-pressure limit can be associated with a first air-flow state. A controller or computing device can transmit commands effecting a first desired air-pressure limit in response to an indicator of a first state. A second desired air-pressure limit can be associated with a second air-flow state. A controller or computing device can transmit commands effecting a second desired air-pressure limit in response to an indicator of a second air-flow state.

For example, the first air-flow state can be exhalation with the associated desired air-pressure limit set as desired positive-end expiratory pressure. For example, the second state can be inhalation, with the associated desired air-pressure limit set as the desired peak inspiratory pressure.

A pressure-limiting valve can be disposed in various locations about a patient interface manifold. For example, such valves can be disposed in an exhalation or exhaust port. A pressure-limiting valve can allow air to escape via an exhaust port when pressure in a cavity reaches a configured level. The exhaust port can vent into atmosphere or into an exhaust system, such as a filter. A filter and/or exhaust system can be advantageously utilized to reduce transmission of pathogens. An exhalation tube can be added to an exhalation port.

Processing circuitry for an actuator can transmit signals to a computing device. Signals can indicate a position of a compression arm and/or a compression force of a spring.

Air-flow sensors can be added to a medical device. For example, an air-flow sensor can detect air flow in an airway of a medical device. An air-flow sensor can associate a detected air flow with a state of operation of a medical device, such as an inspiratory hold, or a patient condition, such as inhalation or exhalation.

Multiple air-flow sensors can detect air flow in multiple airways of a medical device. Multiple air-pressure sensors can associate detected air flows with states of operation of a medical device or patient conditions.

A volume of air delivered in over a determined period can be calculated. A volume of air delivered can be calculated using an average air-flow rate over a period and a duration of a period.

An air-flow sensor can be a differential sensor. An air-flow sensor can be based on an air pressure sensor in a Versamed iVent 201 Adult Disposable Circuit. An air-flow sensor can be an impeller air-flow sensor, hot wire anemometer, ultrasonic air-flow sensor, or other air-flow sensors as known in the prior art.

An air-flow obstruction can be added to an airway. Air pressures in an airway can be measured, such as by placing air-flow sensing ports and associated air-flow meters on either side of an obstruction. Air pressure can be calculated such as by using Bernoulli's equation. A volume of delivered air can be determined, such as by integrating the flow rate over time.

Air flow through an airway can be restricted. Air pressures in an airway can be measured, such as by placing air-flow sensing ports and associated air-flow meters on either side of a restriction. Air pressure can be calculated such as by using Bernoulli's equation. A volume of delivered air can be determined, such as by multiplying a detected air-flow rate and a duration of air delivery.

A patient interface manifold can include powered elements. Powered elements can include, for example, valves and sensors. Powered elements can receive power via peripheral cables. Peripheral cables can be dedicated to powered elements. Alternatively, peripheral cables can deliver power to be used by multiple powered elements.

A patient interface manifold can include a battery module. A battery module can be a power source, for example to be engaged upon a power failure. Backup batteries can be dedicated to high-priority elements. For example, backup batteries can be dedicated to control circuitry and actuators for a pressure-limiting valve. Backup batteries can alternatively be dedicated to sensors, such as air-pressure sensors. A backup battery can provide sufficient power to high priority elements to sustain treatment operation of a patient interface manifold during an interruption in power from a primary source.

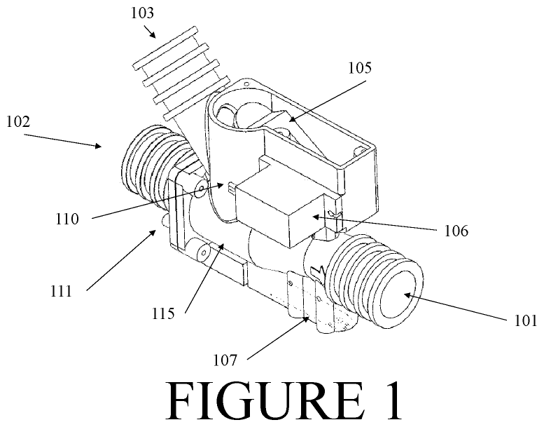

FIG. 1 depicts an exterior oblique view of a patient interface manifold with the spring-valve mechanism exposed. A patient port 101 can be connected to an airway such as an endotracheal tube (not shown). A source port 102 can be connected to a source of positive air-flow (not shown), such as a ventilator or an air blower. Air delivered via a source port 102 can travel through a central chamber 115 before exiting via a patient port 101 into an endotracheal tube. An exhaust port 103 can be a port to atmosphere or connected to an exhalation circuit. Air exhaled into a patient port 101 can travel through a central chamber 115 before exiting via an exhaust port 103.

A spring (not shown) can be compressed by a compression arm 105. An actuator and processing circuitry 106 can cause a compression arm 105 to compress a spring (not shown).

An actuator/controller peripheral cable 110 can deliver power to an actuator and controller circuitry 106. An actuator/controller peripheral cable 110 can transmit signals, such as information about an actuator position from a patient interface manifold to another device, such as a patient computer (not shown). An actuator/controller peripheral cable 110 can transmit signals, such as commands or information to be stored in memory, from another device, such as a patient computer (not shown) to actuator and processing circuitry 106.

A patient interface manifold can include an air-pressure sensor 107. A sensor peripheral cable 111 can deliver power to an air-pressure sensor 107. A sensor peripheral cable 111 can transmit signals from an air-pressure sensor to another device, such as a patient computer (not shown).

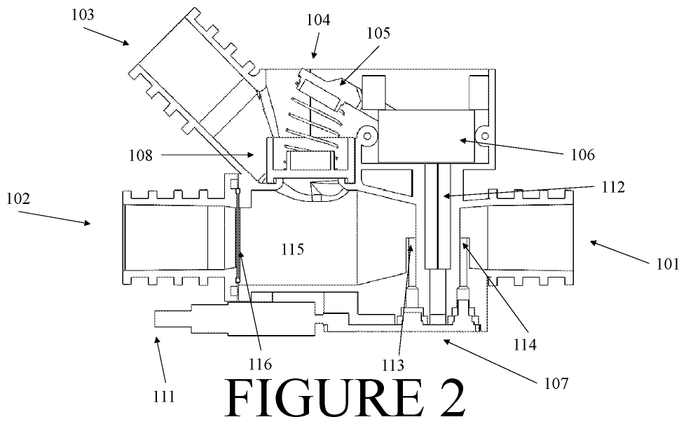

FIG. 2 depicts a cross section of a patient interface manifold.

A spring 104 can exert compression force on a piston 108 to hold it in a closed position. When air pressure in a central chamber 115 multiplied by the area of the piston exceeds the compression force of the spring 104, the piston 108 will move to an open position (not shown). A check valve 116 prevents air in a central chamber 115 from flowing into the positive air-flow source port 102.

An air-pressure sensor 107 can be integrated into a patient interface manifold to detect air pressure in a central chamber 115. An air-pressure sensor can be connected to a first air-flow port 113 and a second air-flow port 114. A first air-flow port 113 and a second air-flow port 114 can be disposed on opposite sides of an obstruction 112 in a central chamber 115. Air can flow laterally around an obstruction 112. Air pressure in a central chamber 115 can be determined using the air flows detected from the first air-flow port 113 and the second air-flow port 114.

An air-pressure sensor 107 can be connected to a patient computer (not shown) via a sensor peripheral cable 111. A signal indicating a detected air pressure can be transmitted to a computing device via a sensor peripheral cable 111.

A sensor peripheral cable 111 can deliver power to an air-pressure sensor 107.

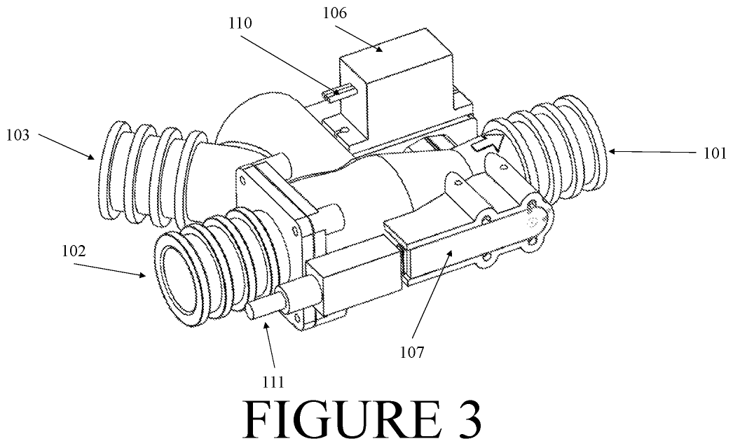

FIG. 3 depicts an underside view of a patient interface manifold. A sensor peripheral cable 111 can be a Universal Serial Bus cable.

In an embodiment, a pressure-regulating valve of a patient interface manifold can be an adjustable pressure-releasing valve. The valve can be a rotary valve. The rotary valve can utilize discs. The discs can be circular.

Each disc can include an aperture. The apertures can be in the shape of a portion of the disc, such as a sector of a circle, a triangle, or a truncated sector cut-off near the center of the disc. The aperture can be in other shapes including irregular shapes, to facilitate advantageous control over volume of air released as the apertures align. An aperture of a disc of an adjustable pressure-releasing valve can have sides aligned with radii of a disc. An angle between sides of an aperture aligned with radii of a disc can be up to 180 degrees, but preferably less than 180 degrees to reduce the possibility of leakage if alignment is imperfect.

A first disc can be coupled with a second disc. The discs can be substantially the same size or different sizes. The discs can have substantially congruent apertures. The apertures can be different shapes.

The discs can be coupled with aligned centers. A first and second disc can be placed to overlap a port of an airway, such as an exhaust airway. A first disc can be placed on an exhaust side of a port of an exhaust airway. A second disc can be placed between an air-flow source and a first disc.

A first disc can be fixed. A second disc can rotate. A first and second disc of an adjustable pressure-releasing valve can be arranged to substantially prevent or reduce air flow through an airway when their apertures are not aligned. O-rings can be used to prevent air from leaking around the edges of the discs.

The discs can be arranged to permit a second disc to rotate. The discs can be coupled to substantially permit or increase air flow through an airway when their apertures are aligned. The discs can be coupled to permit intermediate quanta of air flow through an airway when their apertures are partially aligned. The discs can be pressed sufficiently firmly together to prevent air from escaping between them. The rotation of the discs can be facilitated with lubricant. The discs can be constructed to permit smooth rotation without the need for lubricant, such as with ceramic. The discs can be constructed with self-lubricating materials, such as Teflon.

A second disc can be connected to an actuator by a rotor. An actuator can cause a second disc to rotate. The second disc can be rotated to align, partially align, and unalign the aperture of a second disc with the aperture of the first disc.

An actuator can move the second disc to permit or increase air flow out of an airway to achieve a first desired air pressure in an airway, such as by increasing the extent of alignment of the apertures. A first desired air pressure in an airway can be associated with a first air-flow state. An actuator can move a second disc to prevent or reduce air escape from an airway, such as by reducing the extent of alignment of the apertures. An actuator can move a second disc to prevent air flow out of an airway to achieve a second desired air pressure in an airway. A second desired air pressure in an airway can be associated with a second air-flow state.

The apertures can be aligned with varying degrees. The degree of alignment of the apertures can vary over time, to regulate air pressure at different levels over a period of time.

A check valve can prevent air from flowing in an undesired direction, such as toward an air-flow source during exhalation.

A patient interface manifold can include processing circuitry. Processing circuitry can control an actuator. Processing circuitry can control an actuator to move a second disc to a desired position.

Processing circuitry can include computing logic. Processing circuitry can include computing logic that controls an actuator. Processing circuitry can include computing logic that controls an actuator based on information from a sensor. Processing circuitry can include computing logic to control an actuator to achieve a desired air pressure in an airway, such as by determining whether a desired air pressure has been measured by a sensor.

Processing circuitry can include computing logic to determine a desired position of a second disc relative to a first disc. Processing circuitry can include computing logic to determine a desired degree of alignment of apertures of a first disc and a second disc.

Processing circuitry can include computing logic to control an actuator to regulate air pressure at varying rates over time.

Processing circuitry can include memory storage. Memory storage can include information about multiple desired air pressures. Memory storage can associate desired air pressure with states, such as the state of operation of a medical device or patient conditions. Memory storage can store information about desired air pressures mapped over time. Processing circuitry can include computing logic that controls an actuator according to information in memory storage.

Processing circuitry for an actuator can transmit signals to a computing device. Signals can indicate a position of a second disc and/or a desired air-pressure.

A desired air pressure associated with an air-flow state can be peak inspiratory pressure (“PIP”). An adjustable pressure-release valve can be adjusted to achieve PIP. A source of positive air flow can be connected to a patient interface manifold with an adjustable pressure-release valve. The valve can be controlled to prevent air from escaping from an airway. Air pressure in a chamber can be increased by preventing air from escaping or reducing air escape while the chamber is connected to a source of positive air flow. The valve can be controlled to allow air pressure to increase to a desired air pressure. The desired air pressure can be PIP.

A desired air pressure associated with an air-flow state can be positive-end expiratory pressure (“PEEP”). The valve can be controlled to permit air to escape from an airway until PEEP is achieved. An adjustable pressure-release valve can achieve PEEP while air flows into the airway.

An adjustable pressure-release valve can permit air to release into the atmosphere. An adjustable pressure-release valve can permit air to release into an exhaust airway. An exhaust airway can include a filter.

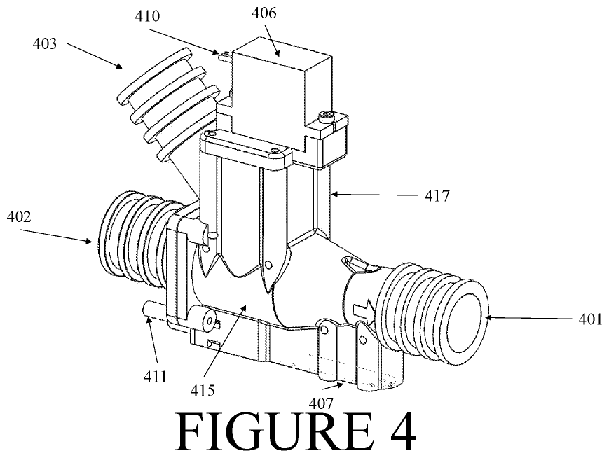

FIG. 4 depicts an oblique view of a patient interface manifold. A patient port 401 can be connected to an airway such as an endotracheal tube. A source port 402 can be connected to a source of positive air-flow, such as a ventilator or an air blower. An exhaust port 403 can be connected to an exhalation circuit. An exhaust port 403 can release direct to atmosphere.

A rotary disc valve (not shown) can be encased in a casing 417 and operated by an actuator and processing circuitry 406.

An actuator/controller peripheral cable 410 can deliver power to an actuator and processing circuitry 406. An actuator/controller peripheral cable 410 can transmit signals to another device, such as a patient computer (not shown) or transmit signals from another device, such as a patient computer (not shown).

A patient interface manifold can include an air-pressure sensor 407. A sensor peripheral cable 411 can deliver power to an air-pressure sensor 407. A sensor peripheral cable 411 can transmit signals from an air-pressure sensor to another device, such as a patient computer (not shown).

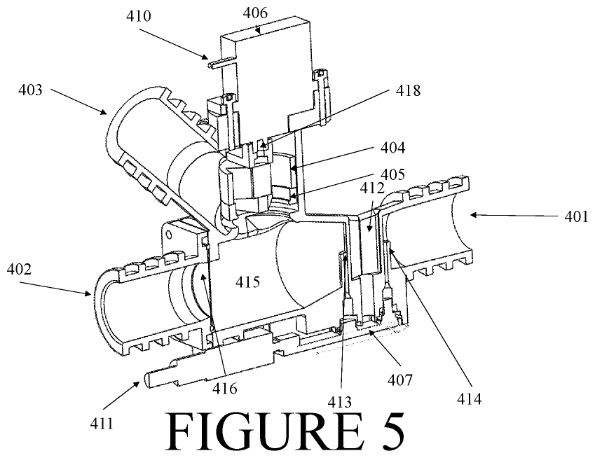

FIG. 5 depicts an oblique view of a cross section of a patient interface manifold.

A rotary disc valve can include a fixed disc 404 with an aperture and a moving disc 405 with an aperture. A position of a moving disc 405 can be moved by an actuator and processing circuitry 406 via a rotor 418. A check valve 416 can prevent air from flowing from a central chamber 415 in an undesired direction.

An air-pressure sensor 407 can be integrated into a patient interface manifold.

An air-pressure sensor can be connected to a first air-flow port 413 and a second air-flow port 414. A first air-flow port 413 and a second air-flow port 414 can be disposed on opposite sides of an obstruction 412. Air pressure in an airway can be determined using the air flows detected from the first air-flow port 413 and the second air-flow port.

An air-pressure sensor 407 can be connected to a patient computer via a sensor peripheral cable 411. A signal indicating a detected air pressure can be transmitted to a computing device via a sensor peripheral cable 411.

A sensor peripheral cable 411 can deliver power to an air-pressure sensor 407.

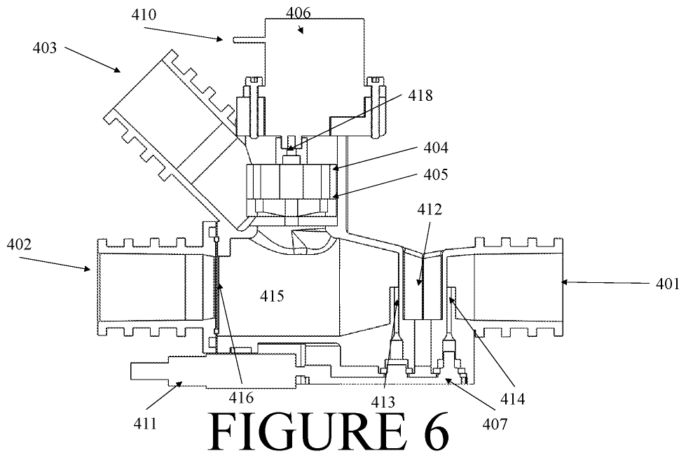

FIG. 6 depicts a cross section of a patient interface manifold.

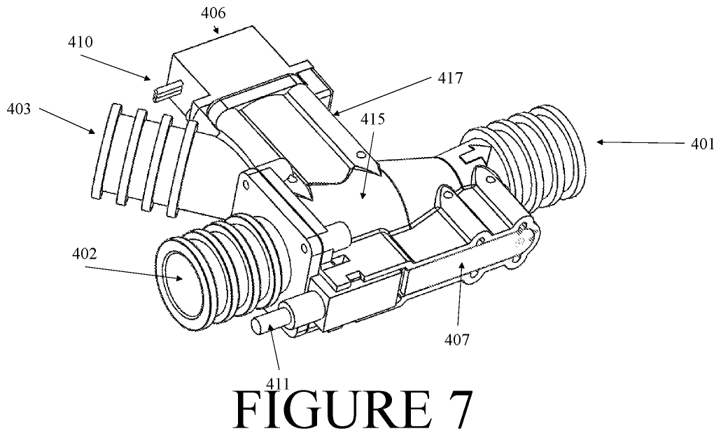

FIG. 7 is an underside view of a patient interface manifold. A sensor peripheral cable 411 can be a Universal Serial Bus cable.

In an embodiment, a patient interface manifold can be symmetrical. A symmetrical patient interface manifold can have ports that are agnostic to directions of airflow.

A symmetrical patient interface manifold can have an air-pressure valve that is agnostic to directions of air flow through a valve. The valve can regulate air-pressures in multiple air-flow circuits, such as an inhalation circuit and an exhalation circuit.

A first disc of a rotary disc valve can be a fixed disc with multiple apertures. The apertures of the first disc can be symmetrically arranged on the disc with an axis of symmetry aligned with an axis of symmetry of the air-flow ports.

The first disc can be coupled with a second disc with one aperture. A first disc and a second disc can be substantially the same size. The discs can alternatively be different sizes. An aperture of a second disc can be substantially congruent to a largest aperture on a first disc. The apertures alternatively can be different shapes.

A rotary disc valve can be placed to overlap apertures of a first and second airway. A first disc having two apertures can be placed so a first aperture faces a first airway and a second aperture faces a second airway.

A first and second disc of a rotary valve can be arranged to prevent air flow from a first and second airway when an aperture of a second disc is unaligned with a first and second aperture of a first disc.

A first and second aperture of a first disc can be substantially congruent, such as to advantageously allow the discs to be agnostic as to orientation. A first and second aperture of a first disc can be different sizes or shapes.

An angle between sides of an aperture of an adjustable pressure-releasing valve for two airways aligned with radii of a disc can be up to 120 degrees. An angle between sides of an aperture of an adjustable pressure-releasing valve for n airways aligned with radii of a disc can be up to 360/(n+1) degrees, but preferably sufficiently less to prevent leakage due to imperfect alignment. The shape of the apertures can be irregular to allow for a scissor action or varying edge, which can advantageously facilitate precise adjustments in air pressure and vary the elasticity of the relationship between rotation of a disc and the area of the open aperture. For example, the aperture of a second disc can have concave curved edges to reduce the amount of area exposed for each aligning increment of rotation of a disc. An aperture of a second disc can be asymmetrical to facilitate different elasticities.

The discs can be arranged to permit air flow via a port of a first airway when an aperture of a second disc aligns or partially aligns with a first aperture of a first disc. The discs can be arranged to prevent air from escaping through a port of a second airway when an aperture of a second disc aligns or partially aligns with a first aperture of a first disc and unaligns with a second aperture of a first disc.

The discs can be arranged to permit air to escape through a port of a second airway when an aperture of a second disc aligns or partially aligns with a second aperture of a first disc. The discs can be arranged to prevent air flow via a port of a first airway when an aperture of a second disc aligns or partially aligns with a second aperture of a first disc and unaligns with a first aperture of a first disc.

A rotary valve can be rotated to release air via a port of a second airway to achieve or maintain a desired air pressure. The valve can achieve a desired air pressure without a countervailing addition of air via a first airway, even if there is a source of positive airflow into a first airway. The valve can maintain peak inspiratory pressure during inhalation. The valve can maintain positive-end expiratory pressure during exhalation. A rotary valve can be rotated to maintain intermediate air pressure conditions. The valve can achieve or maintain desired intermediate air pressure conditions over time.

A first airway can provide a source of air for inhalation, such as from a ventilator. A second airway can provide a repository for air for exhalation, such as to exhaust.

An actuator can control a second disc, such as via a rotor. An actuator can be connected to processing circuitry.

Processing circuitry can include logic to control a second disc to control alignment, partial alignment, and unalignment of a second disc with apertures of a first disc. Processing circuitry can include logic to control a second disc to allow air flow through an aperture of a first disc based on measurements, such as elapsed time, volume of air delivered, or measured air pressure.

Processing circuitry can associate a first aperture of a fixed disc with a first air state and a second aperture with a second air state. The association can be changed.

Air-pressure sensors can be disposed in each segregated chamber of a patient interface manifold. Air-pressure sensors can be disposed in an airway to a patient port of a patient interface manifold.

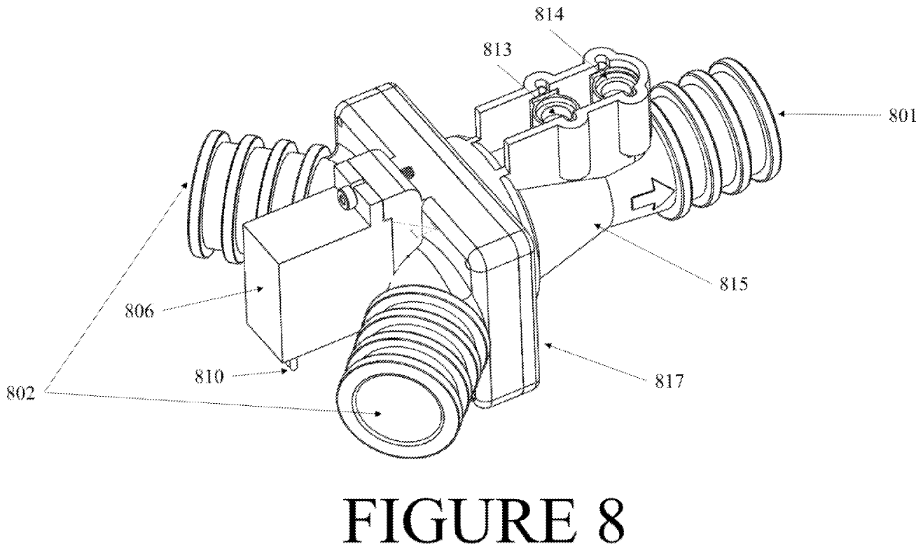

FIG. 8 depicts an oblique view of a symmetrical patient interface manifold with a dual aperture rotary disc valve with circuitry for an air-pressure sensor removed to expose airways for air flowmeters. A patient port 801 can be connected to an airway such as an endotracheal tube. Symmetrical ports 802 can be connected to a source of positive air flow or an exhaust.

A rotary disc valve (not shown) can be encased in a casing 812. An actuator and processing circuitry 806 can control a rotary disc valve (not shown).

A peripheral cable 810 can deliver power to an actuator and processing circuitry 806. A peripheral cable 810 can transmit signals to another device, such as a patient computer (not shown). A peripheral cable 810 can transmit signals from another device, such as a patient computer (not shown).

A patient interface manifold can include an air-pressure sensor (not shown).

An air-pressure sensor can be connected to a first air-flow port 813 and a second air-flow port 814. A first air-flow port 813 and a second air-flow port 814 can be disposed on opposite sides of an obstruction (not shown) in a central chamber 815. Air pressure in a central chamber 815 can be determined using the air flows detected from the first air-flow port 813 and the second air-flow port 814.

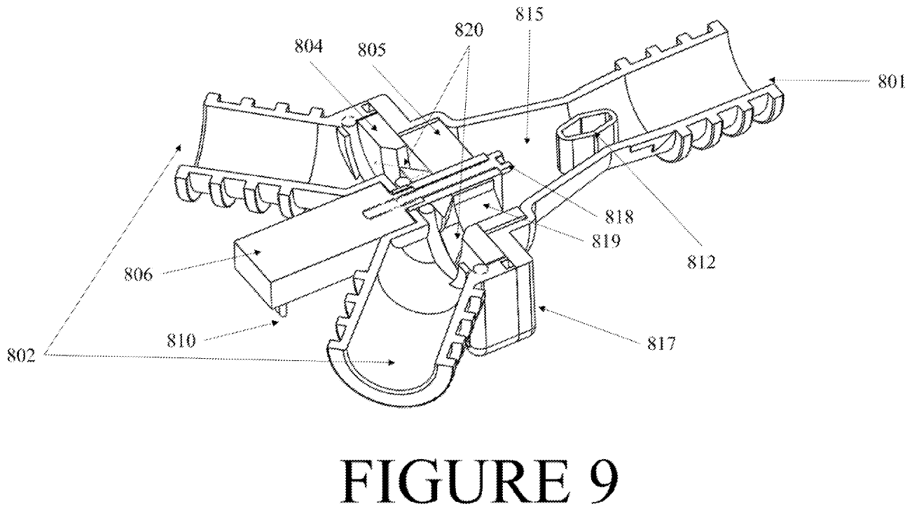

FIG. 9 depicts a horizontal cross section of an oblique view of a symmetrical interface manifold. An obstruction 812 can be integrated into the shape of the patient interface manifold to facilitate measurement of an air-pressure sensor (not shown).

A rotary disc valve can include a fixed disc 804 with two apertures 820 and a moving disc 805 with one aperture 819. A rotary disc valve can be encased in a casing 817. A fixed disc 804 can be placed with apertures 820 arranged to permit air flow to or from one of the symmetrical air-flow ports 802, depending on the alignment of the aperture 819 of the moving disc 805. The position of the moving disc 805 can be changed by an actuator and processing circuitry 806 via a rotor 818 in order to regulate air pressure.

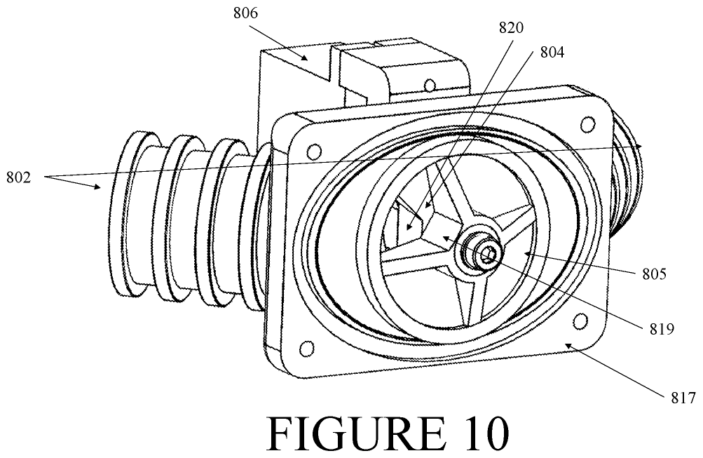

FIG. 10 depicts an oblique view of a rotary disc valve in situs in a symmetrical patient interface manifold.

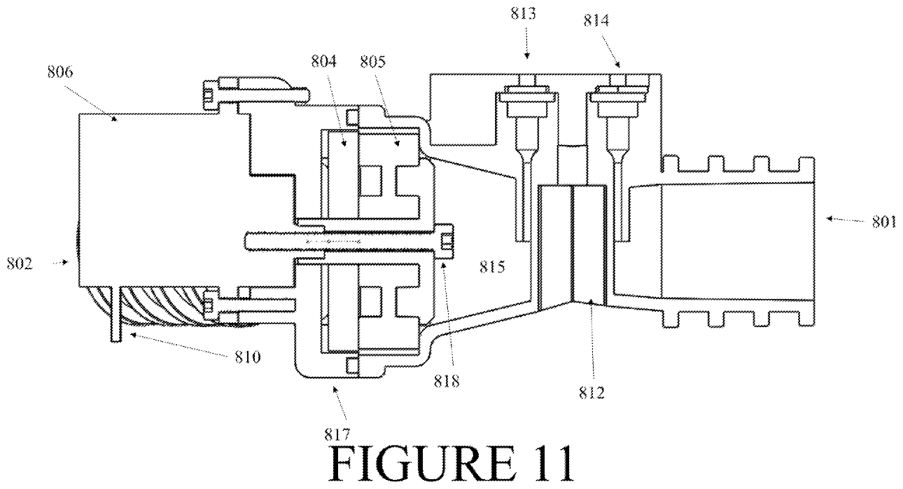

FIG. 11 depicts a vertical cross-section of a symmetrical patient interface manifold

In an embodiment, a patient interface manifold can include multiple chambers. Air pressure in segregated chambers can be regulated by a valve. A pressure-regulating valve can be placed between chambers of a patient interface manifold and a patient port. Chambers can be of a sufficient size to include dedicated air-pressure sensors and other sensors for measuring characteristics of air, such as humidity, oxygen content, or temperature. An airway to a patient port can be of sufficient size to include an air-pressure sensor.

In an embodiment, a patient interface manifold can include processing circuitry that determines which airway is connected to a source of positive air flow. The source of positive air flow can be determined by, e.g., detection of higher air pressure in an airway. Processing circuitry of a patient interface manifold can include computing logic that controls an actuator of a rotary disc valve based on information indicating which airway port is connected to a source of positive air-flow.

A rotary disc valve for a symmetrical patient interface manifold with segregated chambers can include a fixed disc and a moving disc. Alternatively, the valve can operate with only a moving disc, that can be rotated to align the aperture of the moving disc with the airways from either symmetrical port.

A patient interface manifold can include ports for sensor assemblies. Ports can allow access to segregated airways. Ports for sensors can be standardized to accept a variety of sensor assemblies with standardized shapes. Sensor assemblies that conform to standardized ports in a medical device can be modular to allow different combinations of sensors and easy replacement of sensors.

An air-pressure sensor assembly can comprise circuitry for sensing. Circuitry can be included on a printed circuit board. Alternatively, circuitry can be dedicated to sensing.

An air-pressure sensor can comprise an air-flow port body coupled to circuitry. An air-flow port body can include air-flow ports and air-flow tubing. Air-flow port and tubing geometry can be integrated into the shape of the body, such as via embossing, carving, molding, or 3D printing. An air-flow port body can be made of plastic. An air-flow port body can be in the shape of a fin. An air-flow port body can by symmetrical. A symmetrical air-flow port body can be used to reduce error in installation and maintenance.

Air-flow port bodies can be inserted into multiple airways of a medical device, including a positive air-flow source airway, an airway to a patient port, and an exhaust airway.

An air-flow rate can be used to determine a volume of air delivered through an airway. A volume determined from a measured flow rate can be used to determine the volume delivered from a source with unknown or imprecise volume, such as an air blower or compressed air. A volume determined from a measured flow rate can be a redundant check for a device that delivers a known volume of air.

A patient interface manifold can operate agnostic to which airway is connected to a source of positive air-flow. Processing circuitry of a patient interface manifold can determine which airway is receiving positive air flow, such as from air pressures detected from air-flow sensors in each airway. Processing circuitry of a patient interface can control a valve based on a determination of which airway is receiving positive air flow, such as from air-pressure measurements in the airways.

In an embodiment, an air-flow port body can use an obstruction geometry, that can substantially obstruct an airway and present air-flow sensors upstream and downstream of an obstruction.

In an embodiment, an air-flow port body can present multiple air-flow sensing ports into an airway without substantially obstructing an airway, such by using a pitot tube geometry. An air-flow port body can have a first air-flow sensing port. A first air-flow sensing port can face toward a source of positive air flow in a first air-flow state. A first air-flow sensing port can be used to detect air flow from a source of positive air flow in a first air-flow state. A first air-flow sensing port can be used to detect stagnation pressure in a first air-flow state. An air-pressure sensor can have a second port. A second air-flow sensing port can face away from a source of positive air flow in a first air-flow state. A second air-flow sensing port can be used to detect air flow not from a source of positive air flow in a first air-flow state. A second air-flow sensing port can be used to detect static pressure in a first air-flow state.

Air pressure in an air-flow state can be determined. Stagnation pressure and static pressure can be determined from a first air-flow sensing port and a second air-flow sensing port. Air pressure in an air-flow state can be determined using stagnation pressure and static pressure, such as by using Bernoulli's equation.

A second air-pressure sensor can determine air pressure in a second air-flow state. A second air-flow port body can have multiple air-flow sensing ports. A first air-flow sensing port of a second air-flow port body can face toward a source of positive air flow in a second air-flow state. A first air-flow sensing port of a second air-flow port body can be used to detect air flow from a source of positive air flow in a second air-flow state. A first air-flow sensing port of a second air-flow port body can be used to detect stagnation pressure in a second air-flow state. A second air-flow sensing port of a second air-flow port body can face away from a source of positive air flow in a second air-flow state. A second air-flow sensing port of a second air-flow port body can be used to detect air flow not from a source of positive air flow in a second air-flow state. A second air-flow sensing port of a second air-flow port body can be used to detect static pressure in a second air-flow state.

Air pressure in a second air-flow state can be determined. Air pressure in an air-flow state can be determined using stagnation pressure and static pressure, including using Bernoulli's equation. Stagnation pressure and static pressure can be determined from a first air-flow sensing port of a second air-pressure sensor and a second air-flow sensing port of a second air-pressure sensor.

In an embodiment, an air-pressure sensor can be a multi-directional air-pressure sensor. A multi-directional air-pressure sensor can determine air-pressure in multiple air-flow states. A multi-directional air-pressure sensor can have an air-flow port body including multiple ports. A first port of an air-flow port body can face toward a source of positive air flow in a first air-flow state and away from a source of positive air flow in a second air-flow state. A second port of an air-flow port body can face away from a source of positive air flow in a first air-flow state and toward a source of positive air flow in a second air-flow state.

An air-pressure sensor can be coupled to processing circuitry or a computing device. An air-pressure sensor can transmit signals indicating air pressure in an air-flow state. The sensor can transmit signals indicating air-pressure in an air-flow state to processing circuitry or a computing device.

Multiple air-pressure sensors can be coupled to processing circuitry or a computing device. Multiple air-pressure sensors can transmit signals indicating air pressure in multiple air-flow states. Multiple air-pressure sensors can transmit signals indicating air pressure in multiple air-flow states to processing circuitry or a computing device.

A multi-directional air-pressure sensor can be coupled to processing circuitry or a computing device. A multi-directional air-pressure sensor can communicate information about air pressure in multiple air-flow states. A multi-directional air-pressure can communicate indicating air pressure in multiple air-flow states to processing circuitry or a computing device.

An air-flow state can be inhalation. An air-flow state can be exhalation. A first air-flow state and a second air-flow state can be inhalation and exhalation. Multiple air-pressure sensors can measure air-pressure in inhalation and exhalation states. A multi-directional air-pressure sensor can measure air-pressure in inhalation and exhalation states. An air-pressure sensor can communicate information about air-pressure in inhalation or exhalation states. An air-pressure sensor can communicate information about air-pressure in inhalation or exhalation states to processing circuitry or a computing device.

A patient interface manifold can include an array of ports for sensors. The array can reduce the number of patient monitoring devices. The array of ports sensors can be coupled with processing circuitry or a computing device.

A patient interface manifold can include a port for a humidity sensor. A patient interface manifold can include a port for a temperature sensor. A patient interface manifold can include a port for a carbon dioxide sensor. A patient interface manifold can include a port for an oxygen sensor. Ports can be shaped to accept various sensors that conform to the port geometry.

Sensors can detect patient conditions and device operating statuses including: airway pressure (e.g., MIF, MEF, or SNIP), inhalation air-flow rate, exhalation air-flow rate (e.g., PEFR), respiratory rate, respiratory volume (e.g., inspiratory volume, expiratory volume, FVC, FEV1, pressure/volume loop), heart rate, oxygen concentration, air humidity (e.g., hydration, adrenergic activity, CHF), tidal carbon dioxide, air temperature, internal body temperature at baseline or in response to activity (such as based on air/breath temperature or skin temperature). Sensors can be pressed against the skin to measure internal characteristics, such as EEG, ECG, pulse, blood oxygen, blood pressure, body temperature, or other characteristics detectable from the skin.

A patient interface manifold can include processing circuitry, such as a microprocessor. A microprocessor can collect information from sensors. A microprocessor can analyze information from sensors. A microprocessor can transmit information from sensors to a computing device, such as a patient computer. A microprocessor can transmit information to processing circuitry for a valve, such as commands to control a valve. A microprocessor can be included on a circuit board, including a printed circuit board.

A patient interface manifold can include audio devices, such as a speaker or microphones. A speaker can emit sounds such as alarms, indicators of patient conditions, indicators of operating status of a medical device, instructions for patient treatment, instructions for configuring, adjusting, or repairing a medical device, or ambient noise such as music. The sounds can be verbal or non-verbal.

Additional sensors can be included on a patient interface manifold. The sensors can be connected to a processor. Cameras can be connected to a patient interface manifold. Cameras can be directed at a patient airway. The cameras can be used to detect mucosa and soft tissue architecture or the vasculature of the mouth or face to detect blood pressure. The cameras can be used to detect integrity of the patient airway. The cameras can be used to detect changes in patient conditions over time. Cameras can be directed at a patient's external anatomy, such as the face. Cameras can be used to detect pathological and physiological changes. Cameras can be used to detect a change in breathing state, such as a transition from inhalation to exhalation.

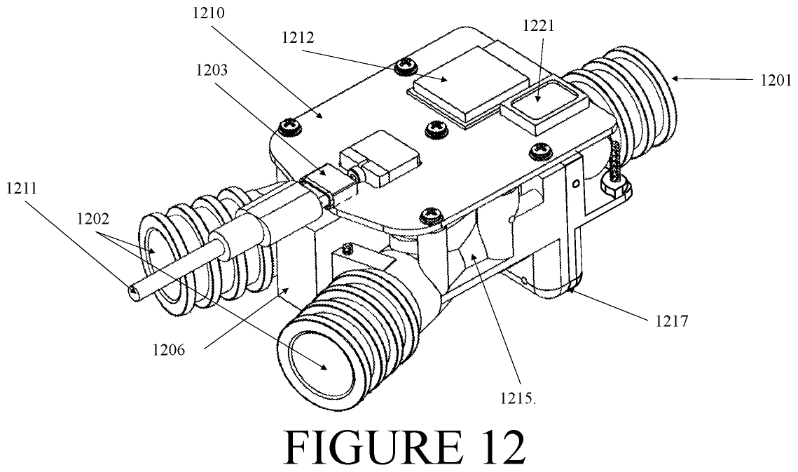

FIG. 12 depicts an oblique view of a symmetrical patient interface manifold with a circuit board included, with the outer casing removed. A patient port 1201 can be connected to an airway such as an endotracheal tube (not shown). Symmetrical ports 1202 can be connected to a source of positive air flow or an exhaust. Each symmetrical port 1202 can be connected to a segregated symmetrical chamber 1215.

A rotary disc valve (not shown) can be encased in a casing 1217. A rotary disc valve (not shown) can be placed at the patient end of segregated symmetrical chambers 1215 to regulate air pressure in each chamber and in a patient airway (not shown). An actuator and processing circuitry 1206 can control a rotary disc valve (not shown).

A printed circuit board 1210 can be added to a patient interface manifold. A circuit board 1210 can connect to actuator and processing circuitry 1206.

A peripheral cable 1211 can connect to a circuit board 1210 via a Universal Serial Bus port 1203. A peripheral cable 1211 can deliver power to a circuit board 1210. A peripheral cable 1211 can transmit signals to another device, such as a patient computer (not shown). A peripheral cable 1211 can transmit signals from another device such as a patient computer (not shown).

A printed circuit board 1210 can include a microprocessor 1212 with computing logic relating to an actuator and processing circuitry 1206 and sensors (not shown), such as to control a position of an actuator.

A patient interface manifold can include a speaker 1221 for transmitting alarms and indicators.

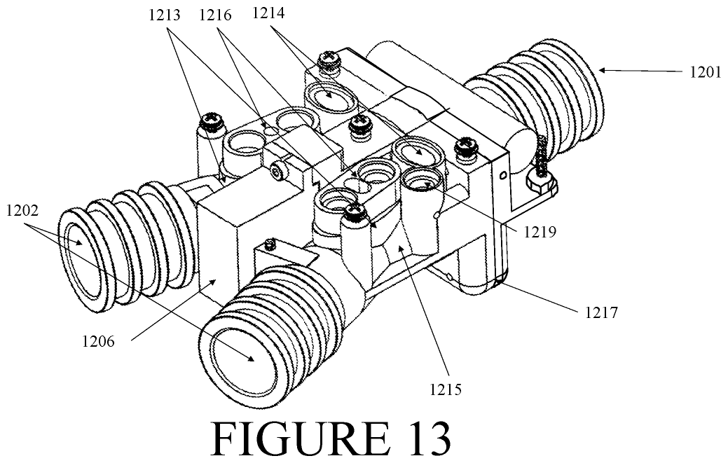

FIG. 13 depicts an oblique view of a patient interface manifold without a circuit board included and without a casing. A patient interface manifold can include an array of ports for sensors including air-pressure sensor ports 1213 for segregated symmetrical chambers 1215, additional accessory ports 1214 that can be for sensors to measure or detect humidity, oxygen content, carbon dioxide content and other conditions, and an air-pressure sensor port 1213 for an airway to a patient port 1201. Symmetrical air-pressure sensor bodies 1216 can be placed into air-pressure sensor ports 1213 to present air-flow ports into segregated symmetrical chambers 1215.

A patient-side air-pressure sensor port 1219 can be provided to detect air pressure in airways on the patient side of a valve. Freeze plugs 1220 can be used to seal the pathway from the air-flow sensing ports (not shown) to the patient-side air-pressure sensor port 1219 as it traverses the casing 1217 for the rotary valve.

Ports can be connected to sensor circuitry on a circuit board (not shown), and signals indicating measurements detected by the sensors can be processed by a microprocessor (not shown) and/or transmitted to a patient computer (not shown).

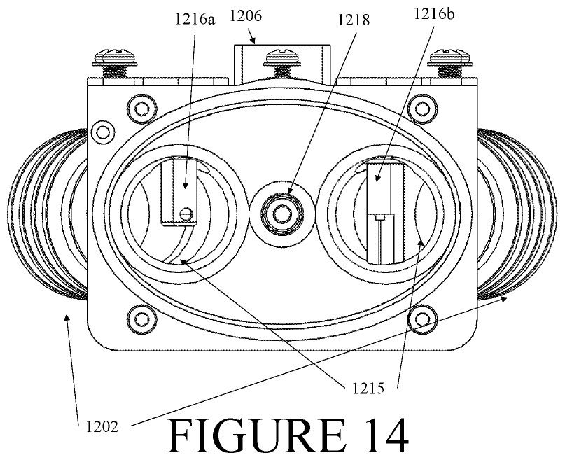

FIG. 14 depicts a vertical cross section of a patient interface manifold facing segregated symmetrical chambers 1215, without the rotary valve. Air-flow port bodies 1216 can be disposed in segregated symmetrical chambers 1215 via air-pressure sensor module ports (not shown). Air-flow port bodies 1216 can have a pitot tube geometry 1216a or an obstruction geometry 1216b. Air-flow port bodies shaped to conform to the shape of the port can be swapped out for bodies of other geometries.

A rotor 1218 can be connected to a moving disc (not shown) of a multiple-aperture rotary disc valve (not shown). An actuator with controlling circuitry 1206 can rotate a rotor 1218 to regulate air flow into the segregated symmetrical chambers 1215.

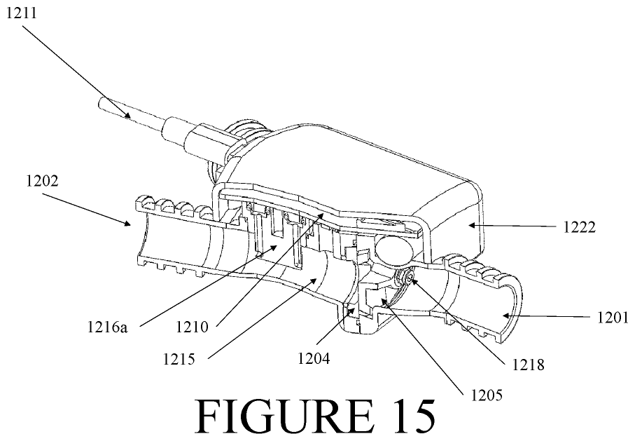

FIG. 15 depicts a vertical cross-section of an oblique view of a patient interface manifold. A casing 1222 can enclose a patient interface manifold. A rotor 1218 can move a moving disc 1205 of a rotary disc valve to align an aperture to allow air to flow in from or out through a segregated symmetrical chamber 1215.

An air-pressure sensor module with a pitot tube geometry 1216a can be presented into a segregated symmetrical chamber 1215 and connected to a circuit board 1210 to detect air pressure in a segregated symmetrical chamber 1215.

Measurements from an air-pressure sensor 1216a can be processed by a microprocessor (not shown) and information based on measurements from an air pressure sensor 1216a can be transmitted to a patient device via a peripheral cable 1211.

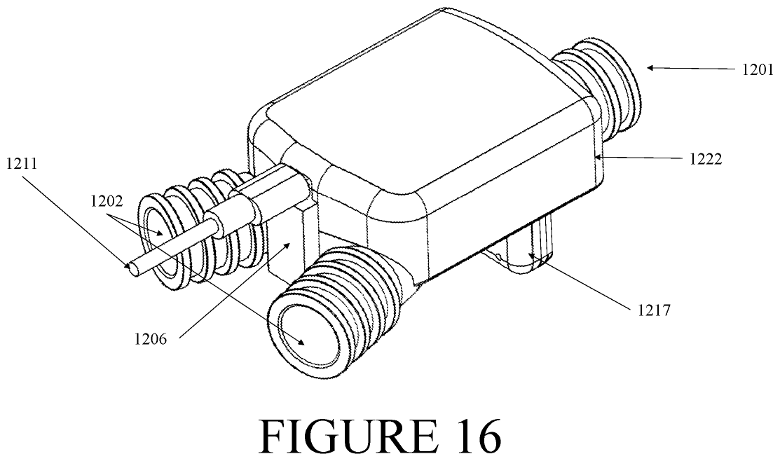

FIG. 16 depicts an oblique view of a patient interface manifold encased in a casing 1222.

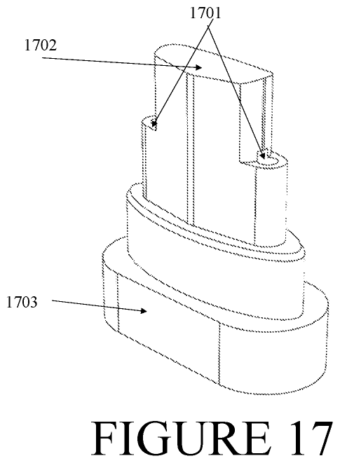

FIG. 17 depicts an oblique view of an air-flow port body with an obstruction geometry.

Air-flow sensing ports 1701 can be disposed on either side of an obstruction 1702. A base 1703 can be a standardized or symmetrical shape to fit in an air-pressure sensor port of a patient interface manifold (not shown).

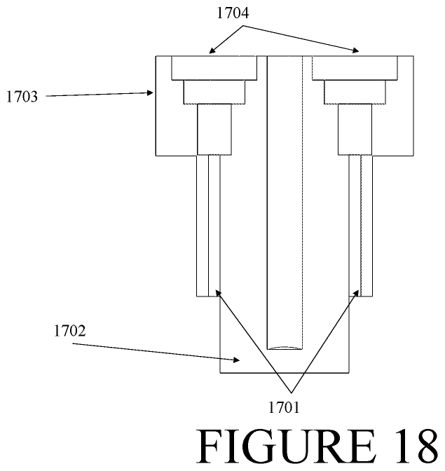

FIG. 18 depicts a cross-section of an air-flow port body with an obstruction geometry.

Air that flows through air-flow sensing ports 1701 can move air-flow indicator mechanisms 1704. Movement of air-flow indicator mechanisms 1704 can be detected and measured by circuitry on a printed circuit board (not shown). The difference in pressure observed via the ports 1701 on either side of the obstruction 1702 is directly proportional to the air flow.

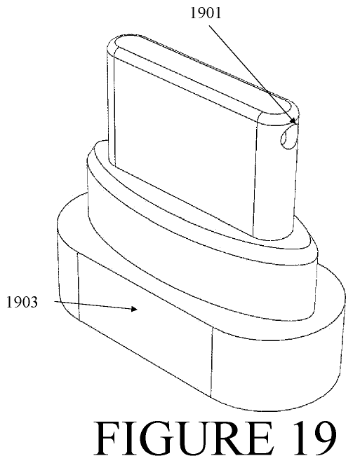

FIG. 19 depicts an oblique view of an air-flow port body with a pitot tube geometry.

Air-flow sensing ports 1901 can be disposed to face in opposite directions on an air-flow port body. A base 1903 can be a standardized or symmetrical shape to fit in an air-pressure sensor port of a patient interface manifold (not shown).

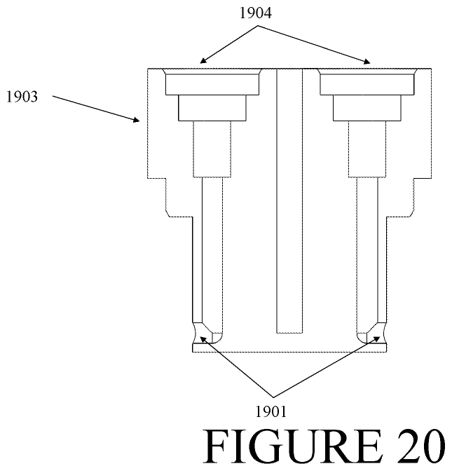

FIG. 20 depicts a cross-section of an air-flow port body with a pitot tube geometry. Air that flows through air-flow sensing ports 1901 (only one shown) can move air-flow indicator mechanisms 1904. Movement of air-flow indicator mechanisms 1904 can be detected and measured by circuitry on a printed circuit board (not shown). Air impinging directly on the port 1901 disposed upstream (facing the source of air flow) causes a pressure rise. Air streaming past the port 1901 disposed downstream (facing away from the source of air flow) is in a static part of the flow resulting in a lower pressure. The flow is proportional to the difference of the two measured pressures.

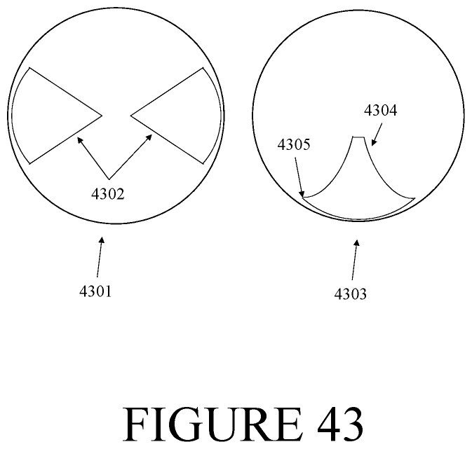

FIG. 43 depicts an alternative geometry for a rotary disc valve for a patient interface manifold. A first disc 4301 can be a fixed disc with two fixed apertures 4302. A second disc 4303 can be a moving disc with one moving aperture 4304. A moving aperture 4304 can have a different shape from the fixed apertures 4302, such as with a varying edge contour 4305 to facilitate precise air-pressure regulation.

A patient ventilator can maintain desired air-pressure states, including peak inspiratory pressure (“PIP”) and positive-end expiratory pressure (“PEEP”).

PIP and PEEP values are typically set with a pressure-regulating valve situated between an endotracheal tube and an airway to a ventilator.

Alternative mechanisms can achieve PIP and PEEP levels without the need for a valve.

It is desirable to reduce “deadspace” or dead volume between the patient and the ventilator, such as in a series of valves and additional tubing. That deadspace typically contains air exhaled by the patient, which has a lower oxygen concentration and higher carbon dioxide concentration than desired. Additionally, the likelihood of a leak or other malfunction increases as more and larger components are used between the ventilator and the endotracheal tube.

A ventilator can be constructed without the use of pressure-regulating valves, such as those typically used to maintain PIP and PEEP. An air blower, such as a high-speed fan or centrifugal blower, can be enclosed in a casing with a single port that can be connected directly to an endotracheal tube. Inhalation and exhalation air can both travel through the blower, without the need for a separate exhalation circuit.

A ventilator can achieve a desired PIP and PEEP by modulating an air speed of an air blower. A speed of an air blower can be modulated by adjusting a speed of an actuator connected to a an air blower. An actuator can be precise and responsive, such as a servomotor, a three-phase permanent magnet motor. or another motor of suitable speed with precise position feedback. An actuator can be controlled by processing circuitry or a computing device.

On inhalation, an air blower speed can be increased until a desired PIP pressure is reached. On exhalation, an air blower speed can be reduced until a desired PEEP pressure is achieved. Alternatively, if no PEEP is required, the air blower can be turned off to allow natural exhalation.

An air-pressure sensor can be added to an air-blower ventilator. An air-pressure sensor of an air-blower ventilator can be a single solid-state differential sensor, such as using a bi-directional pitot tube air-pressure sensor. An air-pressure sensor can be added to a mouthpiece of an air-blower ventilator.

A speed of an air blower can be modulated by processing circuitry. Processing circuitry can include computing logic. Processing circuitry can be mounted on an air blower. Processing circuitry can be connected to a computing device. Processing circuitry can be connected to a computing device via a peripheral cable. Processing circuitry can transmit information to a computing device. A computing device can transmit commands to processing circuitry for an air blower. A computing device can transmit information to processing circuitry for an air blower.

A speed of an air blower can be modulated to effect desired air pressures over time. A speed of an air blower can effect air pressures determined by a model.

Air drawn in via an air blower can be filtered, such as with a HEPA filter.

A patient interface manifold can be connected to an air blower for example, to facilitate or augment pressure regulation, to add sensor capabilities, or to add computer processing capabilities.

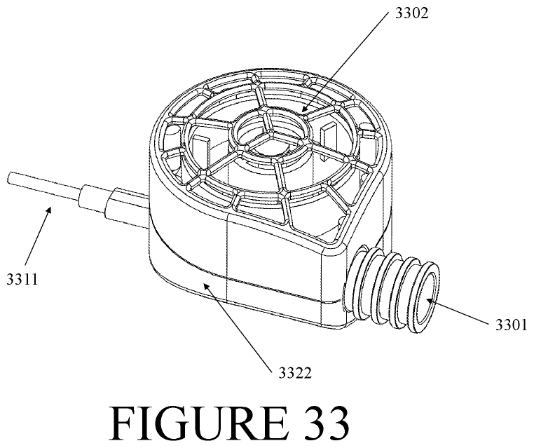

FIG. 33 depicts an oblique view of a direct-to-patient air blower device. A patent port 3301 can be connected to an airway such as an endotracheal tube or directly into a patient's mouth.

Air can be drawn in or exhausted through an open face 3302. An open face 3302 can include a filter (not shown).

A direct-to-patient air blower can be powered via a peripheral cable 3311. A direct-to-patient air blower can send and receive signals via a peripheral cable 3311.

A direct-to-patient air blower can be encased in a casing 3322.

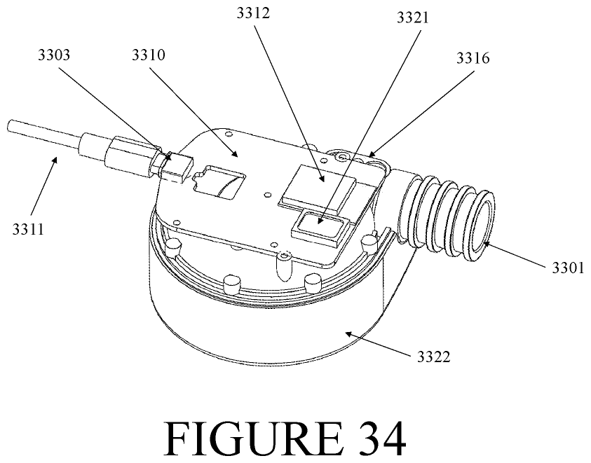

FIG. 34 depicts an underside view of a direct-to-patient air blower device, including a circuit board, with a bottom casing portion removed. A circuit board 3310 can be added to a direct-to-patient air blower. A circuit board 3310 can include a peripheral port 3303 such as a Universal Serial Bus port to receive power and to send and receive signals via a peripheral cable 3311.

An air-flow port body 3316 can be inserted into an airway (not shown) to a patient port 3301 via a sensor port (not shown).

A microprocessor 3312 can be added to a circuit board 3310 with computing logic relating to an actuator and processing circuitry (not shown) and for processing signals from air-flow sensor circuitry (not shown) coupled with an air-flow port body 3316.

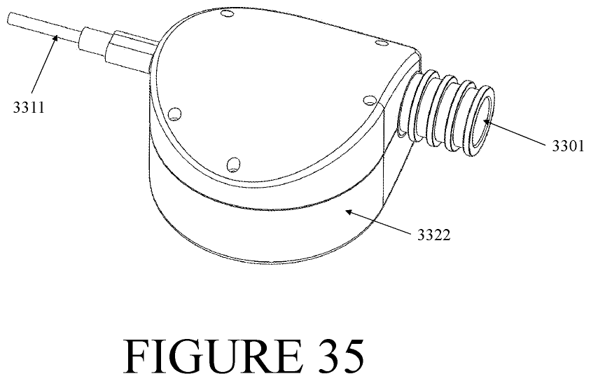

FIG. 35 depicts an underside view of a direct-to-patient air blower device, with the full casing 3322 enclosed.

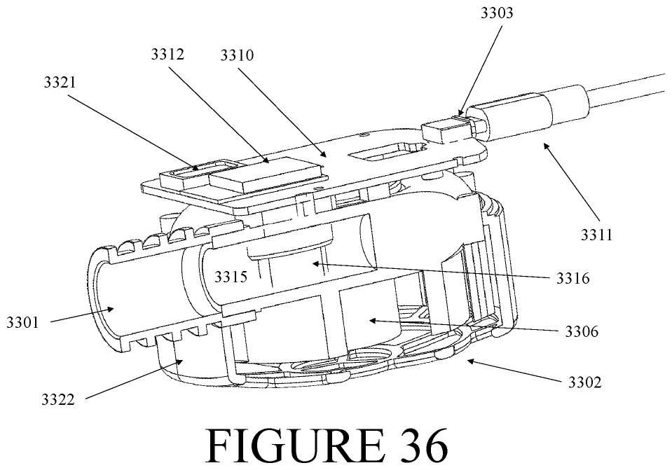

FIG. 36 depicts a vertical cross-section view of a direct-to-patient air blower device, including a circuit board. An air-flow port body 3316 can be extended into an airway 3315 to a patient port 3301 via a sensor port (not shown). An air-flow port body 3316 can be used to detect air pressure in a first air-flow state, such as when positive air flow is toward a patient port 3301 (such as during inhalation) or from a patient port 3301 (such as during exhalation).

An air-blower assembly 3306 can include a centrifugal blower, actuator, and processing circuitry. The air-blower assembly 3306 can be controlled to provide a source of positive air-flow drawing air via an open face 3302, optionally through a filter (not shown). A speed of a centrifugal blower in the air blower assembly 3306 can be controlled to allow exhaled air to escape via an open face 3302, optionally through a filter (not shown). A speed of a centrifugal blower in the air-blower assembly 3306 can be controlled during exhalation to reduce a differential in air pressure between a breathing environment and ambient air pressure or to maintain a desired PEEP level with positive air flow through a patient port 3301. A speed of the centrifugal blower in the air-blower assembly 3306 can be controlled to achieve intermediate PEEP conditions, including multiple PEEP conditions over time. A desired PEEP level can be zero, whereby a centrifugal blower in the air-blower assembly 3306 can be stopped to permit air-flow without a minimum exhalation pressure.

A microprocessor 3312 can transmit signals, such as commands, to an actuator and processing circuitry 3306. A microprocessor 3312 can receive signals, such as information about a position or speed of a centrifugal blower from an actuator and processing circuitry 3306.

The speed of the centrifugal blower in the air-blower assembly 3306 can be modulated to effect air pressure over time mapped to a desired model for cardiopulmonary resuscitation procedures. A user interface, such as a visual screen or audio indicators, can be provided to direct a user to perform chest compressions or other procedures in synchronization with the device's breathing pattern.

A direct-to-patient air blower device can be configured to provide air speeds that effect the air pressure desired for a continuous positive airway pressure device, such as that used for treating sleep apnea or other sleep disorders. CPAP and other modes can be used to, e.g., mitigate altitude sickness or provide a comfortable breathing environment, including for healthy users.

A ventilator that connects directly to an endotracheal tube may generate noise or vibration that can bother a patient, and its mass is typically supported by the patient's body or some other nearby support. A small tubing system can be added to a ventilator such as a ventilator regulated by a valve or an air blower as described above, allowing an air blower to be moved away from the patient's body. A small tubing system can advantageously use an air blower system with minimal deadspace added.

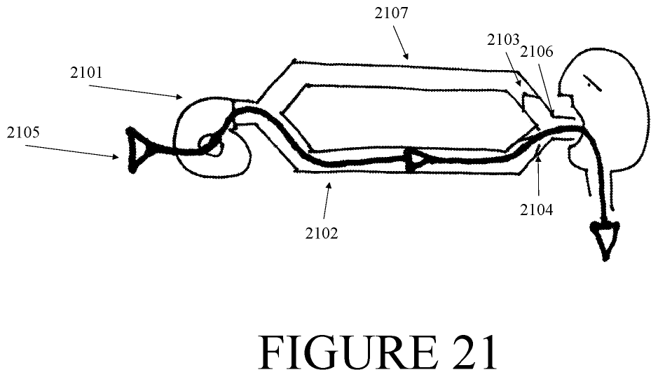

The tubing system can comprise two hoses, joined at each end by a Y-shaped splitter. A first hose can be used for inhalation air flow. A second hose can be used for exhalation air flow. A Y-shaped splitter at one end can include one-way valves for each hose. A valve on an inhalation hose can allow air flow only in the direction from a positive air-flow source to a patient. A valve on a second hose can allow air flow only from a patient to an exhaust. Exhaust can be back into a mouthpiece of an air blower. An air blower can maintain a desired PEEP level with positive air flow through an exhalation tube. FIG. 21 depicts a configuration of an air-blower ventilator circuit with minimal deadspace during an inhalation state. An air blower 2101 can draw air from a source 2105, such as atmosphere, optionally via a filter (not shown). Air can flow through an inhalation tube 2102. A first check valve 2104 can allow air from the inhalation circuit into an airway connected to a patient, such as an endotracheal tube 2106. A second check valve 2103 can prevent air from flowing into an exhalation tube 2107.

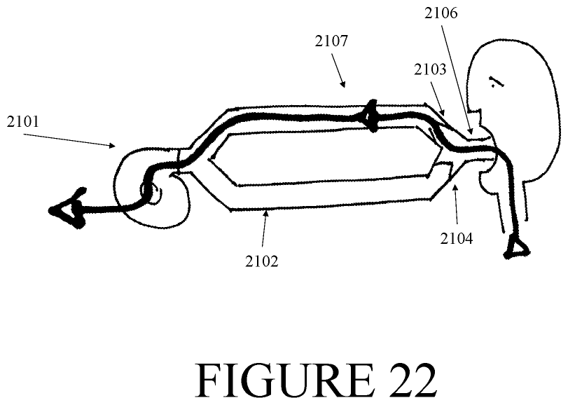

FIG. 22 depicts a configuration of a ventilator circuit with minimal deadspace during an exhalation state. Air exhaled by a patient into an airway connected to a patient, such as an endotracheal tube 2106, can be allowed into an exhalation tube 2107 via a second check valve 2103. A first check valve 2104 can prevent air from flowing into an inhalation tube 2102. Exhaled air can flow into a channel in a ventilator 2101 and into the atmosphere, optionally through a filter (not shown).

In an embodiment, a patient interface manifold can be integrated into a face mask as a diagnostic tool. A mask can have a patient-facing side and an external-facing side. The patient-facing side of a mask can include a sealing member.

A patient interface manifold integrated into a face mask can have multiple air-flow chambers, such as an air-intake chamber and an air-exhaust chambers. The direction of air-flow through a chamber can be regulated, such as by a check valve.

An air-intake chamber can have an air-intake port for air to enter from the environment. A check valve can be integrated into a mask to regulate the flow of air from an air-intake chamber to a patient airway. A patient interface manifold can include sensors to detect chemical and physical characteristics of inhaled air.

An air-exhaust chamber can have an air-exhaust port for air to exit to the environment. A check valve can be integrated into a mask to regulate the flow of air from a patient airway to an air-exhaust chamber. A patient interface manifold can include sensors to detect chemical and physical characteristics of exhaled air.

Processing circuitry, such as a microprocessor, can be contained in a patient interface manifold integrated into a face mask. Processing circuitry can include multiple circuit boards. A circuit board can be associated with an air-flow chamber. Sensors can be disposed in an air-flow chamber to detect information about an air-flow chamber. Processing circuitry can collect information from sensors. Processing circuitry can analyze information collected from sensors. Processing circuitry can transmit signals to another device, such as a mobile device, such as via Bluetooth or TCP/IP. Processing circuitry can transmit signals to another device for use in a designated application. formatted for Signals can indicate information about a patient condition, based on information detected by sensors.

Sensors can be integrated into a mask directly on the patient-facing side to detect patient conditions.

Sensors can be dermal sensors or electrodes for electrocardiogram (“EKG”). Dermal sensors or electrodes can detect EKG signals from contact with an exterior face of a sensor array housing to facilitate patient access. An exterior face of a sensor array housing can include divots to indicate proper placement of patient fingers for two-lead EKG.

A pulse oximeter can be integrated into a mask to interface with a patient's nasal septum.

A microphone can be directed at a patient airway. A microphone can be used to detect the integrity of the patient airway, such as detecting large and small airway obstructions, upper airway obstructions, functional airway changes, disease states, or responses to activity. A microphone can detect the condition of patient lungs. Multiple microphones can be used to differentiate sources of sounds.

Cameras can be integrated into a mask directed at a patient's mouth. A light can be integrated

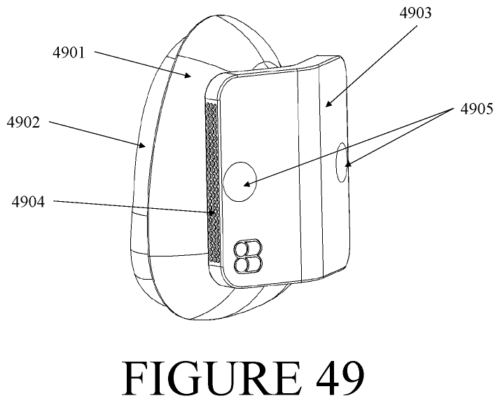

FIG. 49 depicts an oblique external-facing view of a diagnostic tool integrated into a mask with sensor array. A casing 4903 can be integrated into a face mask 4901 having a sealing member 4902. A casing can have an air-intake port 4904 to an air-intake chamber (interior, not shown) and an air-exhaust port (not shown) from an air-exhaust chamber (interior, not shown). Processing circuitry (not shown) can be included in a casing. Electrodes (not shown) can be integrated into the casing that can detect electrocardiogram signals from a patient's fingers (i.e., 2-electrode EKG). Dimples 4905 or other tangible indicators can be integrated into the exterior surface of the casing 4903 to indicate the proper placement of a patient's fingers.

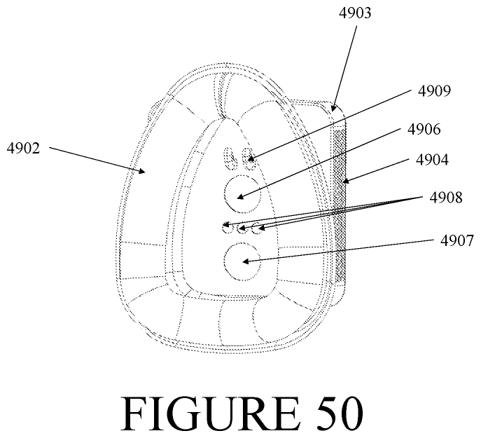

FIG. 50 depicts an oblique patient-facing view of a diagnostic tool into a mask with a sensor array. A sealing member 4902 can maintain a seal against a wearer's face to effect a sealed patient air chamber. A patient-facing side of a mask can have an inhalation check valve 4906 to regulate or modulate the flow of air from an air-intake chamber (interior, not shown) in a casing 4903. A patient-facing side of a mask can have an exhaust check valve 4907 to regulate or modulate the flow of air from a sealed patient air chamber to an air-exhaust chamber and an air-exhaust port in a casing 4903.

A plurality of sensors 4908 such as a microphone or a camera with a light can be integrated unto a mask to measure characteristics about air in a sealed patient air chamber or to measure characteristics about a patient. Sensors 4908 can be communicatively coupled to processing circuitry in a casing 4903. A nasal septum sensor 4909 such as a pulse oximeter can be integrated into a mask and communicatively coupled to processing circuitry in a casing 4903.

A compact ventilator device can be constructed. A compact ventilator device can comprise an air blower and circuitry to control an air blower. Circuitry to control an air blower can include computing logic. Circuitry to control an air blower can include memory or storage.

A compact ventilator device can include a computing device. A computing device can be a patient computer. A computing device can receive signals from sensors. A computing device can transmit signals to processing circuitry for an air blower. A computing device can transmit signals indicating commands for operation of an air blower. A computing device can determine commands based on signals from sensors.

A computing device of a compact ventilator device can provide a user interface, such as a touch screen. A user interface can be used to transmit information to processing circuitry for an air blower.

A compact ventilator device can be connected to a patient interface manifold. A compact ventilator device can operate without being connected to a patient interface manifold.

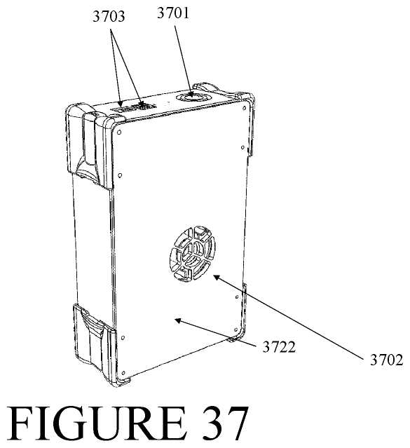

FIG. 37 depicts a rear view of a compact ventilator device. A patient port 3701 can be connected to tubing (not shown) that connects directly to an endotracheal tube or to a patient interface manifold. Air can be drawn from atmosphere via an open face 3702, optionally through a filter (not shown).

A compact ventilator can connect to a computing device or a network via peripheral ports 3703, such as Universal Serial Bus ports or ethernet ports.

A compact ventilator can be encased in a casing 3722.

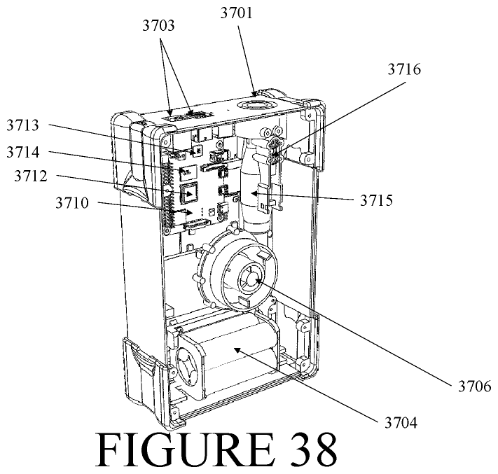

FIG. 38 depicts a compact ventilator without a rear panel. A compact ventilator can include a circuit board 3710. A circuit board 3710 can be a commercially available circuit board such as a Raspberry Pi. A circuit board 3710 can include a microprocessor 3172. A circuit board 3710 can include a peripheral controller 3713 that can control signals transmitted and received via peripheral ports 3703 and transmit them to a microprocessor 3712. A circuit board 3710 can include memory storage circuitry 3714.

A compact ventilator can include an air-blower assembly 3706, including a centrifugal blower, actuator, and processing circuitry. An air-blower assembly 3706 can be used to create a source of positive air-flow into an airway 3715 to a patient port 3701. A speed of a centrifugal blower in an air-blower assembly 3706 can be modulated to effect different air pressures in an airway 3715 to a patient port 3701.

An air-flow port body 3716 can be inserted into an airway 3715 to a patient port 3701. An air-flow port body 3716 can be paired with circuitry for an air-flow sensor (not shown) that can connect to circuitry on a circuit board 3710 to detect air pressure in an airway 3715 to a patient port 3701.

A microprocessor 3712 can include computing logic to control an air-blower assembly 3706 to effect desired air pressures, including based on measurements collected via an air-flow port body 3716.

A compact ventilator can include a power supply element 3704, which can be used as a primary or backup power source.

A compact ventilator can include a user interface (not shown) provided via a microprocessor 3712, storage memory circuitry 3714, displayed via an interface such as an LCD touch screen (not shown).

A source of positive air-flow can be a self-inflating squeeze bag, such as an Ambu bag. Air flow can be automated by placing a squeeze bag in an automated bag squeezer.

An automated bag squeezer can include a constraining mechanism surrounding a squeeze bag. A constraining mechanism can include a plurality of lobes. Lobes can be substantially petal-shaped. Lobes can surround the circumference of a squeeze bag. A constraining mechanism can include four lobes. Lobes can be disposed in pairs surrounding a squeeze bag. Lobes can be disposed to compress a part of a squeeze bag. Lobes can be disposed near the center of a squeeze bag.

A constraining mechanism can include lever-operated fingers. A constraining mechanism can include a notch to allow placing a squeeze bag within a constraining mechanism.

A constraining mechanism can be moved by a movement mechanism.

A movement mechanism can be a bell crank. Rotation of a bell crank can be modulated. The modulation may be from a driven link. The link may be driven by a nut on a threaded motor shaft. Alternatively, the link may be driven by a rolling nut on a smooth motor shaft. A rolling nut can be arranged to permit a desired pitch. A normal force can set a maximum allowable force for a bell crank. A maximum allowable force can control a range of allowable movement for a bell crank in response to a turn of a motor.

A movement mechanism can alternatively be a pair of counter-rotating rings.

A movement mechanism can be moved by an actuator. An actuator can be a motor.

A movement mechanism can be moved to cause a constraining mechanism to compress a squeeze bag. A movement mechanism can be moved to cause a constraining mechanism to uncompress a squeeze bag. The mechanism can be arranged to cause a fixed volume of air to escape a squeeze bag in response to an increment of a motor rotation, with a linear relationship.

An actuator can include a mechanism to indicate a position of a motor, such as a servomotor. An actuator can indicate a position of a motor to processing circuitry. Processing circuitry can include computing logic to determine a volume of air delivered by an incremental rotation of a motor. Processing circuitry can include computing logic to control an actuator to cause an automated bag squeezer to deliver a fixed volume of air from a squeeze bag.

A pitch of a rolling nut can be set to permit movement of 0.01 millimeters to 2 millimeters per turn. A pitch of a rolling nut can be set to permit a complete squeeze of a bag to occur in a range of 20-400 turns of a motor.

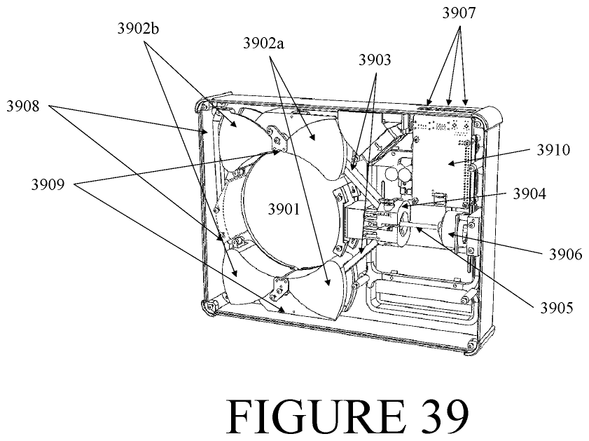

FIG. 39 depicts an oblique cross-section of a bag squeezer with a constraining mechanism in an open position. A shaft 3905 may be turned by a rotor 3906 under control of a circuit board 3910. A rolling nut assembly 3904 is constrained from rotation and so translates in response to the rotation. The translation pulls on connecting rods 3903. The connecting rods 3903 pull on a first pair of petal-shaped lobes 3902a. The lobes pivot about axes defined by pivot assemblies 3909. The rotation causes a closing action of the first pair of lobes 3902a.

An extension (not shown) of the first pair of lobes 3902a constitutes a bell crank to pull on secondary cranks 3908. These in turn pull on a second pair of lobes 3902b. The second pair of lobes 3902b also pivot on pivot assembly 3909. The rotation causes a closing action of the second pair of lobes 3902b.

The geometry of each dimension can be carefully selected to result in a nearly symmetrical motion of all four lobes in a substantially linear relationship with the motion of the rolling nut 3904.

An opening motion is created by a reversal of the shaft and the series of motions in reverse.

A circuit board 3910 can include a microprocessor (not shown) and peripheral controllers (not shown). A circuit board 3910 can be a Raspberry Pi.

A bag squeezer can include peripheral ports 3907.

A bell crank 3904 can be connected to an actuator 3906 via a rotor 3905.

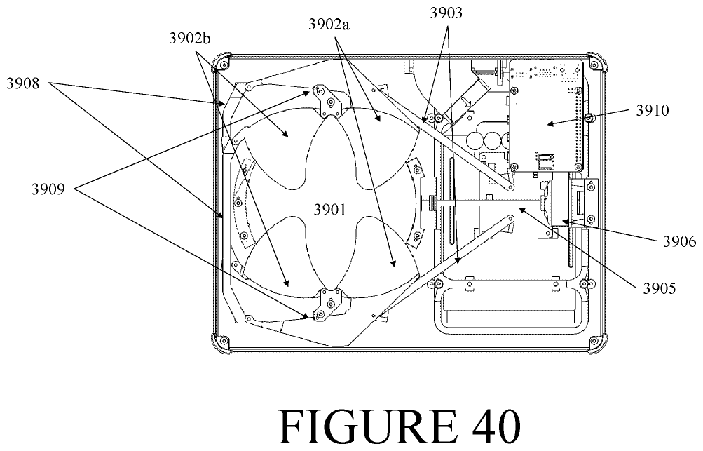

FIG. 40 depicts a cross section of a bag squeezer with a constraining mechanism in a squeezed position. Petal-shaped lobes 3902 can be moved by rods 3903 via levers 3903. Rods can be moved via bell cranks 3908.

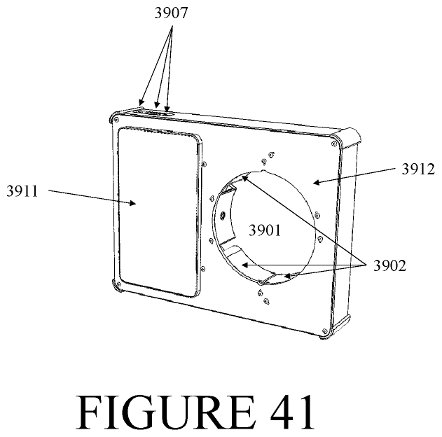

FIG. 41 depicts a bag-squeezer with a constraining mechanism in an open position. A bag squeezer can be encased in a casing 3912. In an open position, petal-shaped lobes can recede into the casing 3912. A user interface, such as a touch screen 3911 can be provided.

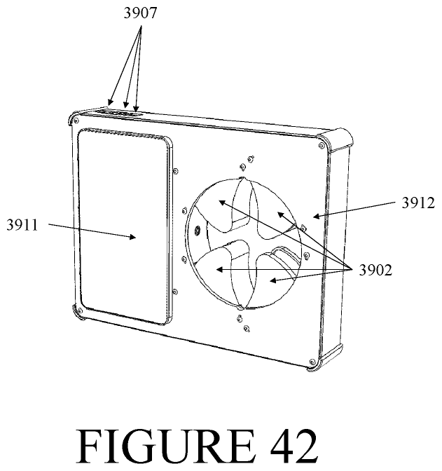

FIG. 42 depicts a bag squeezer in a squeezed position.

Personal protective equipment can provide a separate breathing environment. Personal protective equipment, such as a face mask, can provide a separate breathing environment by sealing against a user's body. A separate breathing environment can be formed by sealing against a wearer's body on the front of the face only (above the nose and below the lips), or alternatively by sealing above the nose and beneath the chin, close to the interface between the bottom of the chin and the neck. Personal protective equipment can provide a separate breathing environment by providing a closed full-body suit.

An air blower system can be integrated with personal protective equipment that provides a separate breathing environment.

An air blower system for personal protective equipment can comprise two air blowers. A first air blower can be a source of positive air flow, into a breathing environment. A second air blower can be a source of negative air flow, out of a breathing environment.

Air blowers can be integrated directly into personal protective equipment. Air blowers can be mounted on the mask on the extreme left or right, advantageously reducing fogging. Air blowers can be connected to personal protective equipment indirectly by hoses, also configured to reduce fogging. A first hose can be associated with a first air blower. A second hose can be associated with a second air blower.

Positive air flow into a breathing environment can be filtered. Negative air flow out of a breathing environment can be filtered. Filters can be tested for contaminants, including viruses such as coronaviruses. Presence of a contaminant in a filter for positive air flow indicates exposure to a contaminant. Presence of a contaminant in a filter for exhaled air indicates presence of a contaminant internal to a user.

An air-blower system for personal protective equipment can include sensors. Sensors of an air flow system for personal protective equipment can communicate with a computing device. A computing device can receive signals indicating measurements of sensors. A computing device can transmit commands to actuators of air blowers. A computing device can include computing logic to transmit commands based on measurements of sensors.

Sensors can measure an air pressure level of a breathing environment. Sensors can measure ambient air pressure.

An air blower system for personal protective equipment can regulate air flow responsive to air pressure of air-flow states.

A sensor can detect a user's inhalation. The first air blower can be accelerated during inhalation. Accelerated air blower speed can reduce a differential in air pressure between a breathing environment and ambient air pressure. The first air blower can be accelerated to prevent a break in a seal of a breathing environment.

A sensor can detect a user's exhalation. A speed of a second air blower can be increased during exhalation. A speed of a second air blower can be increased during exhalation to reduce a differential in air pressure between a breathing environment and ambient air pressure. A speed of a second air blower can be increased to prevent a break in a seal of a breathing environment. This consistency in pressure compared to ambient pressure can advantageously maintain a seal against a body. The actions of the air blowers can effectively neutralize pressures created by the wearer's inhalation and exhalation so as to maintain pressure within the breathing environment near to ambient pressure, advantageously reducing the incentive of the protective equipment to allow a leak in the breathing environment.

Speeds of a first and second air blower can be regulated to permit constant air flow through a breathing environment. Speeds of a first and second air blower can be regulated to improve the freshness and quality of the air in the breathing environment. Speeds of a first and second air blower can be regulated to make breathing feel effortless.

In an alternative embodiment a powered air-blower system for personal protective equipment can comprise one air blower. An air-blower system can be integrated into a patient interface manifold. The patient interface manifold can be constructed as a detachable filter box, surrounding the blower system with filter material. The patient interface manifold can be constructed to fit a standardized or proprietary connecting port for personal protective equipment, such as commercially available full- or half-face masks with attachment ports, such as 3M 6000 series. In a preferred embodiment, a removable battery, such as an 18650 lithium cell battery, can be fitted to each powered air-blower system. Batteries may be readily removed when discharged and replaced quickly with a fully charged battery. Standard batteries and chargers are commonly available.

The patient interface manifold with an air-blower system can include a single solid-state differential air-pressure sensor between the patient and the blower, such as using a pitot tube geometry. The sensor ready communicates with processing circuitry housed in the patient interface manifold. The processing circuitry is capable of generating commands to control and modulate the speed of the air blower.

The processing circuitry can include computing logic to control and modulate the speed of the air blower. The computing logic can include instructions to modulate the blower speed proportional to the air pressure measured by the pressure sensor in the patient interface manifold. The computing logic can include a deadband range in the instructions for controlling the air blower.

A second solid-state differential air-pressure sensor can be disposed between the blower and the filter.

The processing circuitry, the power unit, the blower motor, and the air-pressure sensors can be mounted to a single printed circuit board. The printed circuit board can be exposed to outside air to provide the air-pressure sensors a common “zero reference.”

The computing logic can include high-performance algorithms for controlling the speed and direction of the air blower. The air blower can be modulated in direct proportion to the entire state of the system, which can be determined by the detected air pressures, the speed of the air blower, and their derivatives with respect to time. Gains may be selected by optimizing a cost function including battery life, pressure error, and noise, such as using the well-known gain-selection algorithm, “linear quadratic regulation.”

The state of the system may alternatively be estimated using an optimal estimator, where only some elements of the system are known. An estimator can consider the dynamics of the system, the commands to the air-blowers, and measurement. This kind of estimator is known as a Kalman Estimator.

The processor circuitry of an air-blower system integrated into a patient interface manifold can communicate information wirelessly, such as optically or through a radio frequency connection such as Bluetooth, WiFi, LTE, near-field communication, or 5G. The wireless communication can relay information about the wearer to another device, such as information about breathing rate or breathing depth. Software can analyze information communicated from a patient interface manifold such as to monitor stress or relaxation levels.

In a preferred embodiment the air-blower can be a sensor to detect patient breath activity. Positive air flow from the patient side to the air blower can cause the motor to spin. The motor can be wired as a generator, and resulting voltage can be used to power-up or activate the processing circuitry in the patient interface manifold. Conversely, the air-blower can be configured to power down, enter a low power mode, or deactivate the processing circuitry after a period of absence of positive air-flow from a patient side. The result is device activation can be managed without buttons or switches that add ergonomic hardware and circuitry that add weight and complexity to a patient interface manifold.

Light-emitting diodes may be used to indicate a battery level or to warn of low resources. The blower speed can alternatively be modulated to indicate the state of resources, such as to generate an audible pattern.

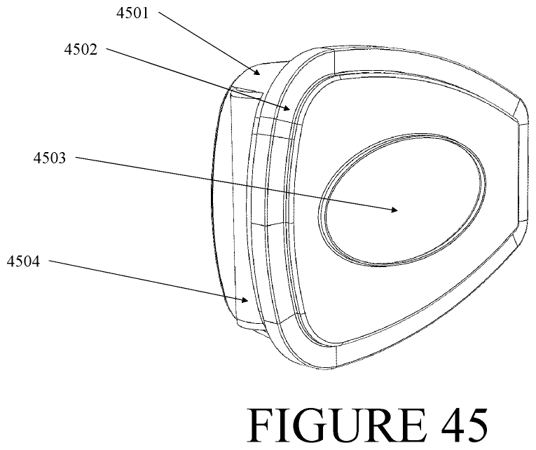

FIG. 45 depicts an oblique exterior external-facing view of a patient interface manifold for connection to a mask. The mask can be a standard commercially available mask with filter attachment or a customized mask. An air-blower system can be integrated into a casing 4501 for a patient interface manifold creating a powered respirator. The replaceable filter medium can be kept in place by a cover 4502. An aperture 4503 in a cover can allow air to flow into or out of the casing 4501. A battery 4504 can be inserted into the casing 4501.

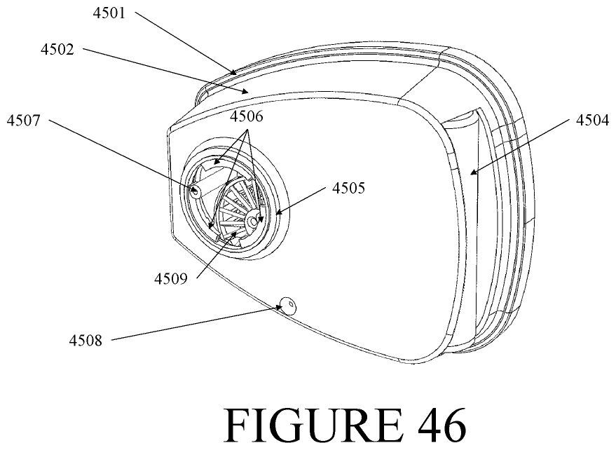

FIG. 46 depicts an oblique exterior patient-facing view of a patient interface manifold for connection to a mask, creating a powered respirator. A casing 4501 can include a patient-facing air-flow port 4505. An air-blower assembly 4509 can impel air from the environment to flow through the patient-facing air-flow port 4505 or draw air through the patient-facing air-flow port 4505 for exhaust to the environment.

A patient-facing air-flow port can have securing means 4506 for securing a patient interface manifold to a mask, such as using a standardized or proprietary securing mechanism. A patient air chamber air-pressure sensor 4507 can protrude through a patient-facing air-flow port 4505 to detect air pressure in a patient air chamber effected by a face mask. A patient air chamber air-pressure sensor 4507 can be communicatively coupled to processing circuitry (not shown). An ambient air-pressure sensor 4508 can be built into the casing 4502.

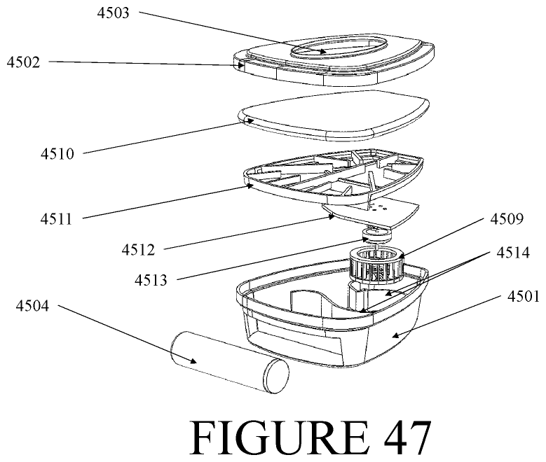

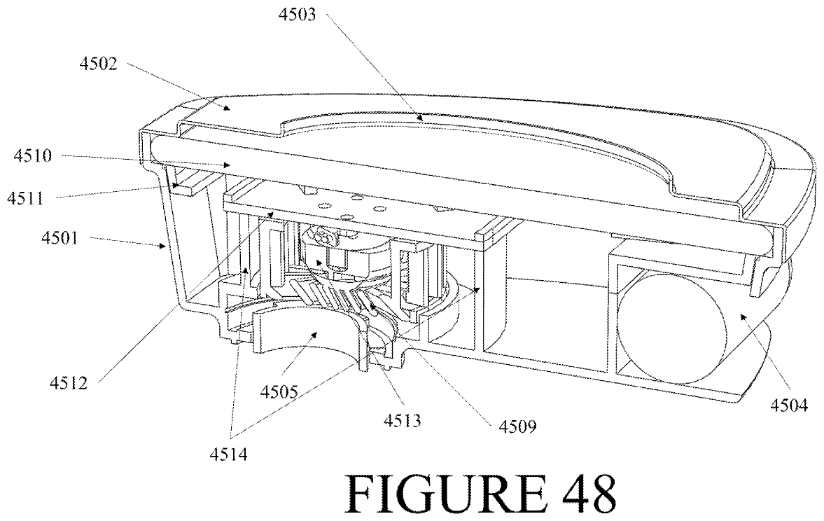

FIG. 47 depicts an exploded view of a patient interface manifold for connection to a mask, creating a powered respirator. Similarly, FIG. 48 depicts a cross-section of the embodiment. A casing 4501 can have securing members 4514 for securing the components of the powered respirator. A powered respirator can have an air-blower assembly 4503 driven by a motor 4513. The motor can be communicatively coupled to a circuit board 4512. A filter manifold 4511 can insert into the casing 4501 to allow unimpeded flow of air to and from the filter medium 4510. A filter medium 4510 can enclose the external-facing face of the casing. A cover 4502 with an air-flow port 4503 can secure the filter 4510 to the casing 4501.

An air blower system can be integrated with a transparent mask. An air blower system for personal protective equipment can provide devices to facilitate verbal communication. An air blower system can include a passive device, such as a speaker cone. An air blower system can include active devices, such as microphones to detect a speaker's voice and speakers to amplify a speaker's voice. Active audio devices can go both ways, both detecting/amplifying the voice of a wearer of a mask and detecting/amplifying external sounds. Active audio devices can connect to external devices, such as to a smartphone via Bluetooth.

An air blower system can include sensors to measure air characteristics, such as flow, air pressure, oxygen content, carbon dioxide content, air temperature, humidity. Measurements can be used to calculate respiratory rate, tidal volume, body temperature, PIP, and other gas-related readings, such as the presence of volatile organic compounds, ammonia, or pH levels.

Sensors can be pressed against the skin to measure internal characteristics, such as EEG, ECG, pulse, blood oxygen, blood pressure, body temperature, or other characteristics detectable from the skin.

Data from sensors can be used to study a wearer's medical condition. Aggregated data from sensors on a respirator system can be used to investigate diagnostic and therapeutic patterns.

An air blower system for personal protective equipment can include sensors to detect when equipment is being worn. A sensor can connect to a program that provides rewards and incentives for wearing a mask over time or in a particular location. Rewards or incentives can include discounts, access to special events, special experiences at a venue, or access to an event at the outset.

A sensor can be used for contact tracing.

A mask can include a camera to detect visible patient conditions.

A mask with an air blower system for personal protective equipment can be designed to facilitate consumption of liquids or food without significant reduction in protection, such as with a straw or other sealing around a delivery device. A respirator system can include ports for nebulized consumables, such as for vaping or other drugs.

A mask can integrate lighting to facilitate views of people's facial expressions and mouth movements, such as to highlight smiles or facilitate lip reading in dark environments. Lighting can serve as a near-field flashlight. A mask can include a display that can display text reflecting what a wearer is saying, directly or in a translated language.

Lighting in a mask can be used to indicate medical conditions or operating conditions of a medical device, such as by changing colors or using flash patterns as alerts.

Lighting in a mask can be used for entertainment purposes, such as for visual effects at a concert or sporting event.

Lighting in a mask can be used as an external signal, such as a distress beacon.

Speeds of a first and second air blower can be regulated to maintain positive air pressure in a breathing environment. A breathing environment This configuration can operate as a respiratory assist device such as a bilevel positive airway pressure (BiPAP) machine.

A face mask can be connected to an air-blower ventilator. The speed of the air blower can be modulated to effect air pressure over time mapped to a desired model for cardiopulmonary resuscitation procedures. A user interface, such as a visual screen or audio indicators, can be provided to direct a user to perform chest compressions or other procedures in synchronization with the device's breathing pattern.

An air blower ventilator can be configured to provide air speeds that effect the air pressure desired for a continuous positive airway pressure device, such as that used for treating sleep apnea or other sleep disorders. CPAP and other modes can be used to, e.g., mitigate altitude sickness or provide a comfortable breathing environment, including for healthy users.

Operation of a mechanical patient ventilator can be automated. A computing device can be connected to the controller of the ventilator. A peripheral cable connecting the controller of the air blower and the computing device can be used to transmit or receive information or commands. The controller of a ventilator can transmit signals indicating various parameters about the device or the patient condition, such as the air pressure of the system, patient heart rate, patient blood pressure to the computing device. The information transmitted by the controller can be collected from sensors embedded in the ventilator or connected to the controller via peripheral cables or wireless connection.

The computing device can transmit commands to the controller of a ventilator to modulate the air pressure. The commands transmitted by a computing device can be determined according to a standard model. A user interface to the computing device, such as a touch screen or smart speaker, can be provided.

The computing device can be built into the medical device directly. Alternatively, the computing device can be an application on another device, such as a smartphone or an internet-connected/cloud device.

A sensor can detect a status of patient conditions or operation of a medical device. Sensors can be added to or integrated with a medical device. Sensors can transmit electronic signals. Sensors can transmit signals indicating measurements taken. Sensors can communicate with a computing device. Sensors can communicate with a computing device via a wired connection. Sensors can communicate with a computing device via a wireless connection. A computing device can control a medical device based on information collected via sensors.

Information about patient conditions or operation of a medical device can be tagged for correlation.

A computing device can transmit signals to other computing devices. Signals transmitted to other computing devices can indicate commands to control a medical device. Signals transmitted to other computing devices can indicate commands to modify stored information used to control a medical device. Signals transmitted to other computing devices can indicate statuses of patient conditions. Signals transmitted to other computing devices can indicate statuses of operation of a medical device.

A computing device can perform analysis of information collected via sensors. Mathematical models can be constructed based on information collected via sensors. A mathematical model can be constructed based on patient-specific information. A mathematical model can also be constructed based on aggregated data from sensors. A mathematical model can be constructed via machine learning.

A computing device can control a medical device using mathematical models maintained or constructed by the computing device. A computing device can modify the operation of a medical device using mathematical models maintained or constructed by the computing device.

Sensors can be air-flow sensors, including pitot tube sensors or obstruction-based sensors. An air-flow sensor can be a thermal sensor.

A pulse oximeter can measure blood-oxygen level. A pulse oximeter can be placed on a patient's finger. A pulse oximeter can be placed on a patient's ear lobe. A pulse oximeter can be placed on a patient's forehead or nasal septum. Changes in blood-oxygen level can indicate a need to modify an oxygen content of delivered air.

Measurements of a pulse oximeter can indicate the level of gas exchange in a patient's respiration.

A temperature sensor can be added to an airway on a ventilator. Thermometers can be added to both the inhalation airway and the exhalation airway. Using the difference in the temperature of air on inhalation versus exhalation, internal body temperature can be determined. This can be achieved with a single thermometer if the ventilator includes an airway used for both inhalation and exhalation.

A dermal sensor can be disposed on a patient's skin. An array of dermal sensors can be disposed on a patient's skin. Dermal sensors can be placed via a patch. Dermal sensors can be placed via a bandage.

Dermal sensors can measure electrocardiogram signals. Dermal sensors can measure respiration rate. Dermal sensors can measure heart rate. Dermal sensors can measure blood sugar level. Dermal sensors can measure blood alcohol content. Dermal sensors can measure blood oxygen level.

A sensor can collect echocardiogram information, including via a dermal sensor. A sensor can be a stethoscope. A sensor can be a blood pressure sensor. A sensor can collect arterial blood pressure. A sensor can collect central venous pressure.

A sensor can detect pressure in a compartment.

Sensors can include esophageal electrodes. Esophageal electrodes can detect diaphragm activity.

Sensors can be body temperature sensors. Multiple body temperature sensors can detect minute differences in the temperature of different parts of a patient's body.

Sensors can detect gas level in blood. Sensors can detect glucose level in blood. Sensors can be ultrasound equipment. Sensors can be bronchoscopy equipment. Sensors can be endoscopy equipment.

Sensors can detect defibrillator activity. Sensors can detect intravenous pump activity. Sensors can detect feeding pump activity.

An array of sensors can provide data about patient conditions from numerous sources. A patient computer can centrally collect data from numerous sources. A patient computer can perform analysis on centrally collected data from numerous sources. A patient computer can present aggregated data to a medical provider. A patient computer can identify relevant data to provide to a medical provider. A patient computer can identify relevant data to provide to a medical provider based on operation of a medical device, such as based on the therapy provided to a patient.

Data provided to a medical provider can facilitate traditional diagnostic methods. Data provided to a medical provider can facilitate the development of new diagnostic methods. Data provided to a medical provider can facilitate the implementation of new diagnostic methods. Data provided to a medical provider can facilitate the development of new strategies for monitoring patient status. Data provided to a medical provider can facilitate the development of new strategies for adjusting operation of a medical device or other treatment mechanisms, such as changing posture, administering medication, or other treatment independent of the operation of the device.

A patient computer can automatically adjust the operation of a medical device. A patient computer can automatically adjust operation of a medical device based on aggregated patient data. A patient computer can automatically adjust operation of a medical device reactive to patient conditions. A patient computer can automatically adjust operation of a medical device proactively.

A patient computer can collect data about patient conditions correlated with adjustments to operation of a medical device.

Sensors can indicate a plurality of patient conditions. Sensors can indicate a plurality of medical device operations. Data from sensors can indicate changes in patient conditions or medical device operations.

Data from sensors can be collected in a repository. A repository can be linked to patient electronic medical records.

Aggregation of information about patient conditions can reduce the number of devices needed to monitor patient conditions. Patient conditions can be inferred from aggregated data about other patient conditions. Patient conditions can be inferred from different sources of aggregated data about other patient conditions. Inferences about patient conditions from differing sources can be used as verification. Inferences about patient conditions can be used to verify direct measurements of patient conditions. Differences between inferred patient conditions and direct measurements of patient conditions can indicate anomalies in operation of a device. Differences between inferred patient conditions and direct measurements of patient conditions can indicate anomalies in underlying patient conditions. Differences between sensor data and a mathematical model can imply changes in a patient condition or a problem with a device.

Information about operation of a medical device can be aggregated. Information about operation of medical devices can be correlated with aggregated information about patient conditions.

Aggregation of information about patient conditions or operation of medical devices can be used to automate documentation of patient conditions. Aggregation of information about patient conditions or operation of medical devices can be used to automate medical record keeping.

Information about patient conditions and device operation can be aggregated. Aggregated data can be used to research relationships among patient conditions and device operations. Aggregated data can be used to improve device functionality. Aggregated data can be used to model natural physiological function. Aggregated data can be used to improve a device to better effect patient conditions similar to natural physiological function. Aggregated data can be used to examine disease processes. Aggregated data can be used to recognize disease processes. Aggregated data can be used to diagnose disease. Aggregated data can be used to improve diagnostic tools. Aggregated data can be used to determine effective treatment strategies.

A software operating thread can be dedicated to an air-pressure limiting valve.

A controller or a computing device can be powered by a standard peripheral cable. A standard peripheral cable can be a USB cable.

The implementations herein can include general-purpose computers, processors, microprocessors, hardware accelerators, software accelerators, servers, cloud-based technology (generically referred to herein as computers where context allows), or combinations thereof. The computer can have internal memory or external memory for storing data and programs such as an operating system (e.g., Linux, iOS, Windows 2000, Windows XP, Windows NT, OS/2, UNIX, etc.) and one or more applications. Applications can include, for example computer programs implementing the techniques described herein, authoring applications (e.g., programs for word processing, databases, spreadsheets, simulations, and graphics) capable of generating documents or other electronic content, client applications (e.g., an Internet Service Provider (ISP) client, an e-mail client, or an instant messaging (IM) client) capable of communicating with other computer users, accessing various computer resources, and viewing, creating, or otherwise manipulating electronic content; and browser applications (e.g., Microsoft's Internet Explorer, Google Chrome, Mozilla Firefox, and Apple Safari) capable of rendering standard Internet content and other content formatted according to standard protocols such as the Hypertext Transfer Protocol (HTTP), HTTP Secure, or Secure Hypertext Transfer Protocol.

The computing devices herein can include one or more central processing units (CPUs) for executing instructions in response to commands from executable code sent via communication devices for sending and receiving data. Examples of communication devices include an internal bus, a modem, an antenna, a transceiver, a router, a dish, a communication card, a satellite dish, a microwave system, a network adapter, and/or other mechanisms capable of transmitting data and/or receiving data, whether wired or wireless. In some embodiments, the processors can be graphics processing units (GPUs) or graphics accelerators. In preferred embodiments, tensor processing units (TPUs) are implemented. TPUs are relatively recent advancements originally designed for artificial intelligence accelerator application-specific integrated circuits (ASICs) developed by Google for neural network machine learning. The computers can also include input/output interfaces that enable wired and/or wireless connection to various peripheral devices. The peripheral devices can include a graphical user interface (GUI) and/or remote devices. A processor-based system of the computer can include a main memory, preferably random-access memory (RAM), or alternatively read-only memory (ROM), and can also include secondary memory, which can be any tangible computer-readable media. Tangible computer-readable medium memory can include, for example, hard disk drives, removable storage drives, flash-based storage systems, solid-state drives, floppy disk drives, magnetic tape drives, optical disk drives (e.g., Blu-Ray, DVD, CD drive), magnetic tapes, standalone RAM disks, etc. A removable storage drive can read from or write to a removable storage medium. As will be appreciated, removable storage media can include computer software and data.

An actuator can be controlled by controller or processing circuitry. Processing circuitry can include computing logic. Processing circuitry can receive signals from sensors. Processing circuitry can receive signals from sensors. Processing circuitry can include computing logic that controls a device based on signals from sensors.

Processing circuitry can receive signals that indicate an air pressure in an airway. Processing circuitry can include computing logic that controls a device based on an air pressure in an airway.

Processing circuitry can include memory storage. Memory storage can include information about desired air pressures over time. Processing circuitry can include computing logic that controls an actuator according to information in memory storage.

Memory storage can store information indicating desired air pressures. Processing circuitry can include computing logic that controls a device based on stored information indicating desired air pressures.

Processing circuitry can include computing logic to control an actuator to achieve a desired air pressure in an airway. Processing circuitry can include computing logic to control an actuator to move a valve to achieve a desired air pressure in an airway. Processing circuitry can include computing logic to control an actuator to move a centrifugal blower to achieve a desired air pressure in an airway.

Memory storage for processing circuitry can store information associating desired air pressures with air-flow states. Memory storage for processing circuitry can store information associating desired air pressures with a variable. A variable can be associated with desired air pressures for air-flow states. A variable can be time.

Information in memory storage for processing circuitry can be modified. Stored information can be modified in response to a signal from a computing device.

Processing circuitry can receive signals from a computing device. A computing device can transmit signals to a controller circuitry. A computing device can transmit signals to controller circuitry indicating a desired air pressure. A computing device can transmit signals to controller circuitry indicating desired air pressures over time.

Processing circuitry can include commercially available processing hardware. Processing circuitry can include common microcontrollers, such as Arduino, STM32, ATmega32u2, or ATmega32u4. Processing circuitry can be compatible with commercially available processing hardware, including, for example, Arduinos. Processing circuitry can be a printed circuit board.

Processing circuitry of a medical device can communicate with a computing device.

A patient computer can be a computing device associated with a medical device. A patient computer can be co-located with a patient using a medical device. A patient computer can be a tablet computer. Alternatively, a patient computer can be an internet-connected/cloud device, accessible via a general-purpose computer, such as a tablet, smartphone, or computer. A patient computer can be a processor directly integrated into a medical device.

A patient computer can receive signals indicating measurements by sensors of a medical device or multiple medical devices. A patient computer can receive signals indicating measurements directly from sensors. A patient computer can receive signals indicating conditions of a patient. A patient computer can receive signals indicating operating statuses of a medical device. A patient computer can store information based on signals received.

A patient computer can communicate with a medical device without requiring internet connectivity, such as via Bluetooth, near-field communication, wired connections, such as USB, or an isolated local area network.

A patient computer can connect to a remote computing device. A remote computing device can connect to a network, such as the internet. A remote computing device can connect to telemedicine software. A remote computing device can transmit information from a patient computer.

A patient computer can be built from a suitable commercially available computer part, such as a Raspberry Pi. Software for a patient computer can be based on open source projects, such as Raspberry Pi, Electron, React, React Native, BLHeli, or Node.js.

A case can be provided to adapt a commercially available tablet for use as a patient computer associated with a medical device. A case can include connectivity adapters. A case can include a supplemental power supply.

A patient computer of a medical device can connect to a remote computing system. Multiple patient computers of medical devices can connect to a remote computing system. A remote computer system can be on-site at a medical care facility. A remote computer system can be off-site from a medical care facility.

A remote computing system can aggregate data from multiple patient computers. A remote computing system can maintain information about the status of multiple patient computers, such as the patient associated with the patient computer, operating status, repair needs, software version, or power level.

A remote computing system can deliver software updates to patient computers. A user of a remote computing system can schedule software updates to patient computers, based on factors such as available downtime, review of software updates, or urgency.

In an embodiment, a remote computing system can be on-site at a care facility, including a server or a personal computer connected to a local network. A medical provider on premises of a care facility can use an on-site remote computing system to communicate with multiple patient computers from a centralized interface. An on-site remote computing system can provide a reliable connection to patient computers, independent of any Internet downtime or congestion. An on-site remote computing system can be backed up on premises.

An on-site remote computing system can collect data from multiple patient computers at a medical care facility. An on-site remote computing system can analyze data from multiple patient computers at a medical care facility. An on-site remote computing system can analyze data reflective of quality of service delivered by a care facility. An on-site remote computing system can transmit information, such as alarms or updates, from a patient computer to staff connected to a care facility's system. An on-site remote computing system can store information that associates staff to a patient device. An on-site remote computing system can transmit information from a patient device to staff associated with a patient device.

An on-site remote computing system can be backed up to an off-site computing system, such as via the Internet. A computer on premises can monitor access to a patient area. A computer on premises can control access to a patient area.

In an embodiment, a remote computing device can be off-site from a care facility. An off-site remote computing system can be used to collect data from multiple patient computers. An off-site remote computing system can be used to collect data from multiple on-site remote computing systems.

An off-site remote computing system can be a computing system for a network of facilities or an affiliate of a care facility, such as Kaiser Permanente. An off-site remote computing system can be a computing system for a device manufacturer. An off-site remote computing system can be a computing system for an industry organization. An off-site remote computing system can be a computing system for a data analytics tool.

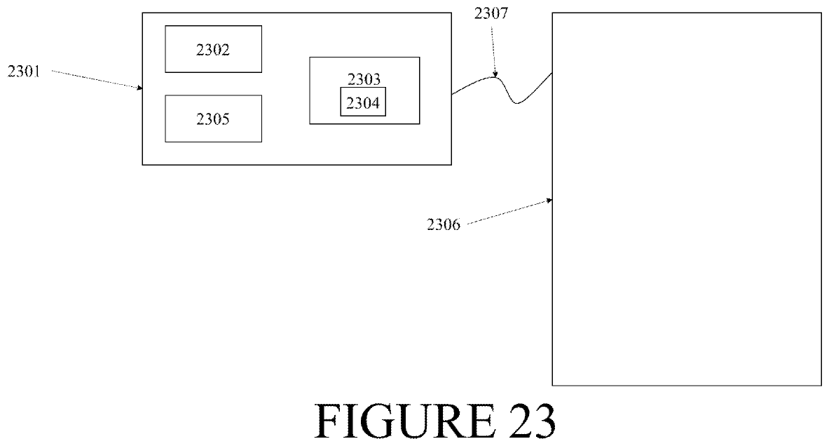

Aggregated data of a remote computing system can be anonymized or pseudonymized. Data can be anonymized or pseudonymized before transmission from a patient computer and a remote computing system. Data can be anonymized or pseudonymized within a remote computing system. Data can be correlated by anonymous or pseudonymous identifying tags. FIG. 23 depicts a block diagram of a medical device 2301 connected to a patient computer 2306. A medical device 2301 and a patient computer 2306 can be connected by a communications link 2307. The communications link 2307 can be a wired or wireless connection.

A medical device 2301 can include an actuator 2302. The actuator 2302 can move components of a medical device 2301 to deliver treatment to a patient (not shown). Processing circuitry 2303 can control an actuator 2302. Processing circuitry 2303 can control an actuator 2302 based on signals from a patient computer 2306. Processing circuitry 2303 can transmit signals indicating operation of a medical device 2301 to a patient computer 2306.

Memory storage 2304 for controller circuitry can store information about operation of a medical device 2301. Processing circuitry 2303 can control an actuator 2302 based on information in memory storage 2304, including based on stored computing logic. Information in memory storage 2304 can be changed based on signals from a patient computer 2306.

A sensor array 2305 can collect information about patient conditions. A sensor array 2305 can transmit signals indicating information about patient conditions to controller circuitry 2303. A sensor array 2305 can transmit signals indicating information about operation of a medical device 2301 to controller circuitry 2303. A sensor array 2305 can transmit signals indicating information directly to a patient computer 2306. A sensor array 2305 can transmit sensor data via controller circuitry 2303. A sensor array 2305 can transmit signals about patient conditions or operations of a medical device.

Processing circuitry 2303 can include computing logic to control an actuator 2302 based on signals from a sensor array 2305. Processing circuitry 2303 can include computing logic to change information in memory storage 2304 based on signals from a sensor array 2305.

A patient computer 2306 can aggregate information about operation of a medical device or patient conditions from a medical device 2301. A patient computer 2306 can aggregate information about operation of a medical device or patient conditions from processing circuitry 2303 of a medical device 2301. A patient computer 2306 can aggregate information about operation of a medical device or patient conditions from a sensor array 2205 of a medical device 2301.

A patient computer 2306 can transmit signals to a medical device 2301 based on aggregated information from a medical device 2301. A patient computer 2306 can transmit signals to processing circuitry 2303 to control an actuator 2302 of a medical device 2301. A patient computer 2306 can transmit signals to processing circuitry 2303 to change information in memory storage 2304. A patient computer 2306 can transmit signals to processing circuitry 2303 to change computing logic.

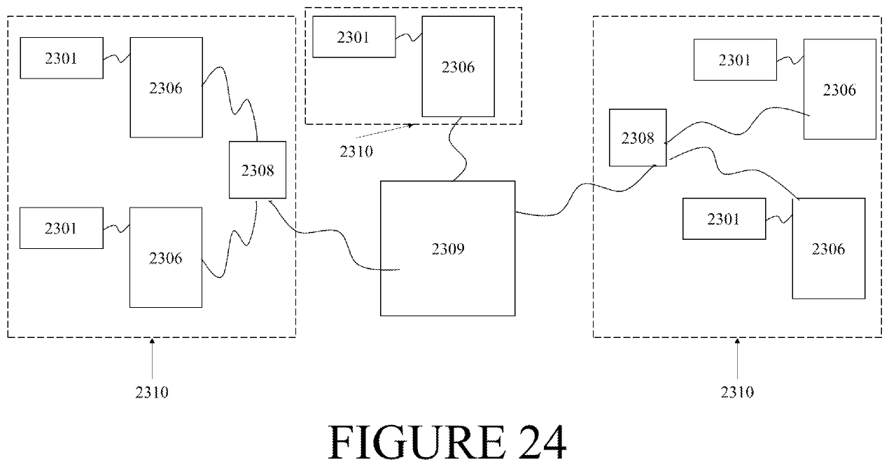

FIG. 24 depicts network topologies for medical devices 2301, patient computers 2306, on-site remote computing systems 2308, and off-site remote computing systems 2309.

Patient computers 2306 can connect to an on-site remote computing system 2308. An on-site remote computing system 2308 can connect to an off-site remote computing system 2309. An on-site remote computing system 2308 can transmit information from patient computers 2306 and medical devices 2301 in a site 2310 with an on-site remote computing system 2308 to an off-site remote computing system 2309.

Patient computers 2306 can connect to an off-site remote computing system 2309. Patient computers can transmit information from a site 2310 without using an on-site remote computing system 2308.

A mathematical model can be constructed to model a patient condition. A model can mathematically model multiple patient conditions. A model can be used to detect a patient condition, including a pathological or physiological state. A model can be used to detect a patient condition not directly measured by a sensor. A model can be used to predict a future patient condition. A model can be used to provide a user interface layout associated with a use case.

A model can be a standard model. A model can be a standard model sensitive to basic information, such as a patient's age, weight, or height.

A model can be based on information collected from sensors. A model can be constructed using information collected from sensors of a medical device. A model can be constructed using information collected from sensors of a medical device associated with one patient. A model can be constructed using information collected from sensors of medical devices associated with multiple patients. A model can be constructed using information collected over time. A model can be constructed using information from sources other than sensors, such as electronic medical records, treatment history, the timing of administration of particular medications, all of which can be compared and correlated with sensor data.

A model can be based on relationships between patient conditions and operating modes of a medical device. A model can be based on relationships between patient conditions and operating modes of a medical device across time, such as by correlating a first patient condition, a first operating status of a medical device, a second operating status of a medical device, and a second patient condition.

A model can be based on patient conditions, operating modes of a medical device, and user interface layouts. A model can predict user interface layouts likely to facilitate user interactions to improve patient conditions.

Sensors can detect changes in patient conditions. Sensors can detect changes in patient conditions that deviate from a model.

A deviation from a model can indicate a diagnosis. A deviation from a model can indicate a need for medical intervention.

A deviation from a model can indicate an error in a model. A deviation in a model can indicate an error in a data-collection mechanism.

A model can be constructed using machine learning. A model can be modified using machine learning.

A model can be a state-space model.

A model can be constructed based on simulations of changes in patient conditions.

Lungs can be modeled. A model can relate to a specific patient's lungs. A model can include parameters relating to elasticity, mass, and damping in a patient's lungs. A model that addresses the dynamics of this system may can facilitate determination or estimation of the actual state of a particular patient's lung system in view of measurements taken. Optimally, an estimator or model will consider both system dynamics and any noise detected by sensors.

A model related to lungs can be based on information indicating air-pressure. A model related to lungs can be based on information indicating air-pressure in an inhalation airway. A model related to lungs can be based on information indicating air-pressure in an exhaust airway. A model related to lungs can be based on information indicating air-pressure in an endotracheal tube airway.

A model related to lungs can be based on information indicating air temperature. A model related to lungs can be based on information indicating air temperature in an inhalation airway. A model related to lungs can be based on information indicating air temperature in an exhaust airway. A model related to lungs can be based on information indicating air temperature in an endotracheal tube airway. A model related to lungs can be based on patient internal temperature.

A model related to lungs can be based on information indicating air oxygen content, information indicating heart rate, echocardiogram information, information indicating respiratory rate, and/or information about operation of a one or more ventilators. A model relating to lungs can be based on a PEEP setting of a ventilator, a PIP setting of a ventilator, and/or information about operation of a ventilator over time. A model relating to lungs can be based on a combination of information collected from sensors and information about operation of a ventilator, information collected from sensors following changes in operation of a ventilator, information about patient conditions following changes in operation of a ventilator, an indicator of an improvement of patient conditions following changes in operation of a ventilator, and/or an indicator of a deterioration of patient conditions following changes in operation of a ventilator.

A model can relate to any patient condition, pathology, or physiology, including heart conditions, blood conditions, nervous system conditions, digestive conditions. A model can address patient conditions during anesthesia, unconsciousness, or comatose.

A user interface for a medical device can provide options to select operating modes.

A medical device can provide programmable operating modes. Computing logic can be provided to determine an operating mode for a medical device based on conditions. Conditions can be threshold measurements of sensors.

A programmable operating mode can be responsive to sedative interval periods.

A patient computer can determine an operating mode based on basic patient information. A patient computer can determine an operating mode based on patient conditions. A patient computer can determine an operating mode based on measurements of patient conditions from sensors and basic patient information. A patient computer can determine an operating mode using a model.

A patient computer can proactively modify an operating mode based on changes in patient conditions.

A patient computer can reduce injuries to a patient by proactively modifying an operating mode.

A patient computer can provide notifications regarding automated changes to an operation of a medical device. A user of a medical device can modify a frequency of notifications. A user of a medical device can modify a scope of notifications.

Software to control a medical device can prevent disruption of operations of a higher priority by lower-priority operations.

A main computing thread in a patient computer can generate separate threads dedicated to tasks or collections of tasks. Each additional thread can be assigned priority levels. A first priority level can be assigned to tasks that are critical to the operation of the device, such as therapeutic operations. Lower priority levels can be assigned to other tasks like maintaining a user input display or aggregating and analyzing collected data. If computing resources become strained, lower-priority tasks can be permitted to fail to permit the provisioning of sufficient resources to continue the higher priority operations.

Controlling circuitry of a medical device can include computing logic to maintain operations without intervention from a patient computer. Controlling circuitry of a medical device can include computing logic to adjust operations responsive to signals from sensors.

A computing device for a medical device, such as a patient computer, can provide a user interface. A user interface can be a touch screen. A user interface can be used to control operation of a medical device.

A user interface can display numerous rows of data and control options. Information can be presented in portrait mode. For example, the user interface in portrait mode can display control options and data simultaneously. A user interface can present a menu on startup. A menu can present a choice among operating modes. A user interface can require dual confirmation from a user before accepting a change in operation.

A user interface can comprise delineated sections. For example, a first delineated section can be associated with controls for a ventilator device. A second delineated section can be associated with feedback. Feedback can relate to a patient condition or status, and can relate to operation status of a device. A user interface can use visual indicators to indicate categories of information in delineated sections. A first delineated section can use a light tone. A second delineated section can use a dark tone.

A user interface can use navigation designs for delineated sections. A user interface can use different navigation designs for delineated sections. A user interface can use navigation designs suitable for information to be displayed in delineated sections. A delineated section can display a fixed set of information. A delineated section can change the information it displays in response to a user action, such as scrolling or swiping a delineated section, pressing a button, or issuing a voice command.

A user interface can present options to change parameters that can be changed independent of other parameters. A user interface can present options to change parameters that can be changed independent of other parameters without detrimental effect to a patient or causing technical errors. For example, a user can set an inhalation-exhalation ratio or a respiratory rate.

An inhalation-exhalation ratio can be set via a single slider. An inhalation-exhalation ratio can be set without requiring a user to simultaneously adjust inhalation time and exhalation time or requiring an undesirable or invalid control setting while other control settings are brought into alignment.

Parameters can have presets. These can be advantageous, allowing for example quick adjustment of parameters. Presets can be defined according to common values. Presets can be defined according to models based on basic patient data, such as age and weight. Presets can be configured and tuned by a user and can be displayed alongside a precise setting interface.

Parameters can be set to precise inputs input via a keypad.

Parameters can be configured to respond to changes in other parameters. A user interface can present an option to set a target fraction of inspired oxygen. A user interface can display an air-flow rate associated with a target fraction of inspired oxygen. A user interface can indicate an action required by a medical provider to achieve a target fraction of inspired oxygen, such as setting an air-flow rate on an air-flow source. A user interface an indicate action to be taken with a visual signal, such as flashing a required setting in pink. A user interface can take automated action to effect a desired air-flow rate to achieve a target fraction of inspired oxygen.

A user interface can permit an inspiratory hold maneuver. A user interface can permit an inspiratory hold maneuver in an “advanced” menu.

A user interface can include an option to pair a ventilator device to a remote computing device. A remote computing device can be a mobile phone or a personal computer. A user interface can use a QR code or other common and well-known pairing mechanism, such as a web interface and unique alphanumeric code or Bluetooth, to pair a ventilator device to a remote computing device.

A user interface can include an option to pair a ventilator device to a remote software suite. A remote software suite can be an application, such as a telemedicine application. A user interface can use a QR code or other common and well-known pairing mechanism, such as a web interface and unique alphanumeric code or Bluetooth, to pair a ventilator device to a remote software suite.

A user interface can be adjusted for particular use cases. An emergency user interface can be provided for use by emergency responders. In such cases, it can be advantageous to present a limited number of options on the interface. Further improvements, for an emergency user interface can be implemented, such as only providing options that require limited computational and power requirements.

A different user interface can be provided for longer-term applications. The different interfaces can be implemented through software configurations allowing for a different user experience from the same hardware interface. In other words, aspects of the user interface can be preprogrammed for adapting to specific uses as desired. A long-term user interface can provide more granular options. A long-term user interface can provide options focused on long-term patient care.

A patient computer can determine specific use cases. For example, the determination can be based on information about a patient condition. A patient computer can determine a use case based on information about operation of a medical device. A patient can determine a use case based on a model.

A patient computer can associate a user interface layout with a use case. A patient computer can provide a user interface layout in response to information indicating a use case. A patient computer can modify a user interface layout.

A patient computer can measure patient conditions correlated with user interface layouts. A patient computer can provide a user interface layout based on a model.

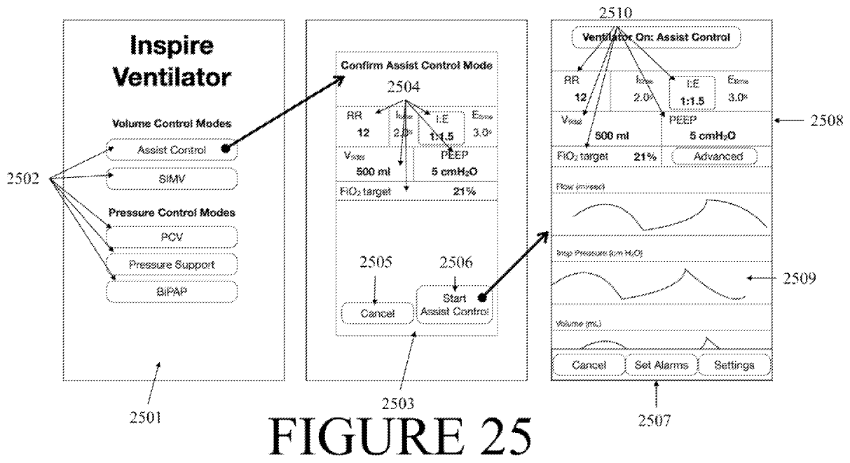

FIG. 25 depicts a series of perspectives, screens, and windows for a user interface for configuring a ventilator mode. Here, the user interface can be connected to or embedded in a ventilator device (not shown) and can display a home screen 2501 upon startup, following any initialization process. The home screen 2501 provides buttons 2502 indicating operating modes for a ventilator device.

Following an interaction with a button 2502, a user interface can display a control menu window 2503. The control menu window 2503 can present control options 2504 associated with desired device operation settings, such as respiratory rate, inspiratory-expiratory ratio, tidal volume, PEEP, or target fraction of inspired oxygen. A control menu window 2503 can present information responsive to changes in control options based on default settings, such as inspiratory time and expiratory time based on inspiratory-expiratory ratio and a default respiration time.

A control menu perspective 2503 can include a cancel button 2505. A control menu window 2503 can include a start button 2506. A start button can require dual confirmation before starting operation according to input via control options 2504.

The user interface can provide an operating screen 2507. The user interface can display an operating screen 2507 following confirmation of settings via a control menu perspective 2403.

An operating screen 2507 can include, for example, a first delineated section 2508 and a second delineated section 2509. The first delineated section 2508 can display control settings of a ventilator device 2510 such as those discussed above. The second delineated section 2509 can display feedback related to a patient condition, physiology, pathology, or status, such as air flow, inspiratory pressure, volume of delivered air, present respiratory rate, or respiratory rate measured over the past minute. The second delineated section 2509 can be adjusted in response to user interaction to display different feedback information.

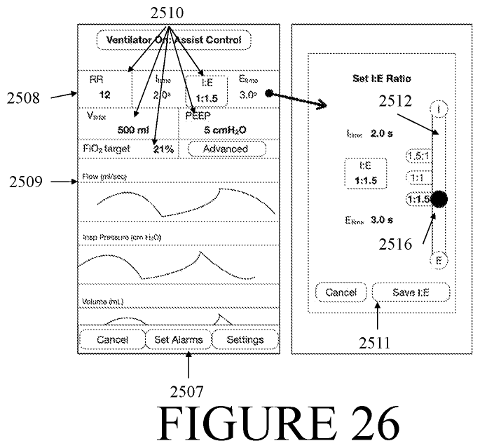

FIG. 26 depicts a series of perspectives, screens, and windows depicting a change of an operating setting, namely inspiratory:expiratory ratio. A user can interact with a control setting 2510 in a first delineated section 2508 of an operating system, such as by touching on a touch screen. Following an interaction, a control-setting window 2511 can be displayed.

A control-setting window can permit change of a setting, such as inspiratory:expiratory ratio via an input mechanism, such as a slider 2512 with a handle 2516. Each control setting 2510 can relate to an operating setting that can be changed without requiring a change to another control setting 2510. Secondary settings 2513, such as inspiratory time and expiratory time can be automatically adjusted responsive to a change in a control setting 2510, such as inspiratory:expiratory ratio.

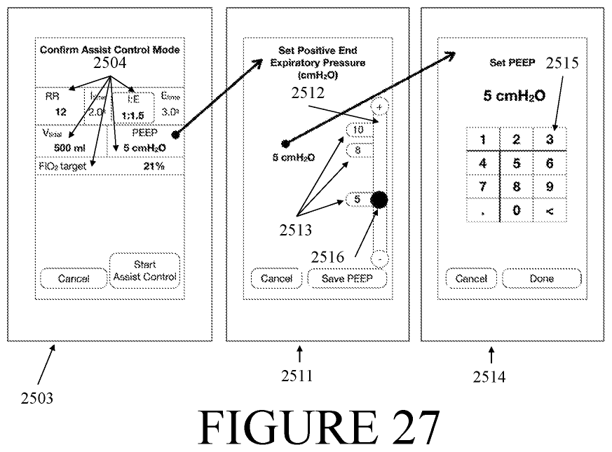

FIG. 27 depicts a series of perspectives, screens, and windows depicting a change of an operating setting, namely a positive-end expiratory pressure parameter. A control menu window 2503 can present control options 2504 associated with device operation settings with common values. A user can interact with control options 2504. Upon interaction, a user interface can present an individual control-setting window 2511. An individual control-setting window can include a slider 2512. Preset values 2513 can be displayed near a slider 2512 to indicate an area of a slider associated with a preset value 2513. A handle 2516 on a slider 2512 can snap to a nearest preset value 2513 to facilitate precise use of preset values.

A user interface can display a precise control setting window 2514. A precise control setting perspective can present a keypad 2515 for entering precise values for a control setting.

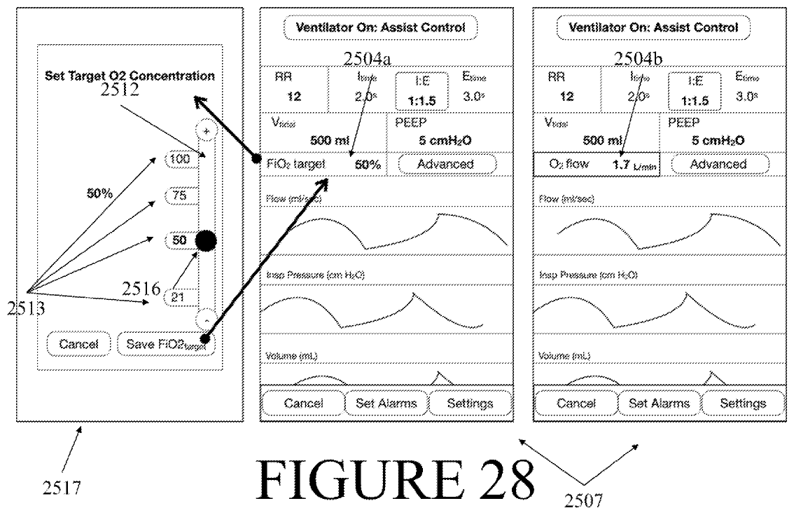

FIG. 28 depicts a series of perspectives, screens, and windows depicting a mechanism for setting a target fraction of inspired oxygen for a device that lacks direct oxygen control. In response to a user interaction with an oxygen setting, a target oxygen concentration setting window 2517 can be displayed. A user can use a slider 2512 and a handle 2516 to select a target oxygen concentration.

An operating screen 2507 can alternate displaying a target oxygen concentration or fraction of inspired oxygen control option 2504a and a required oxygen flow level 2504b, indicating a need to set an oxygen flow level directly at an oxygen source (not shown).

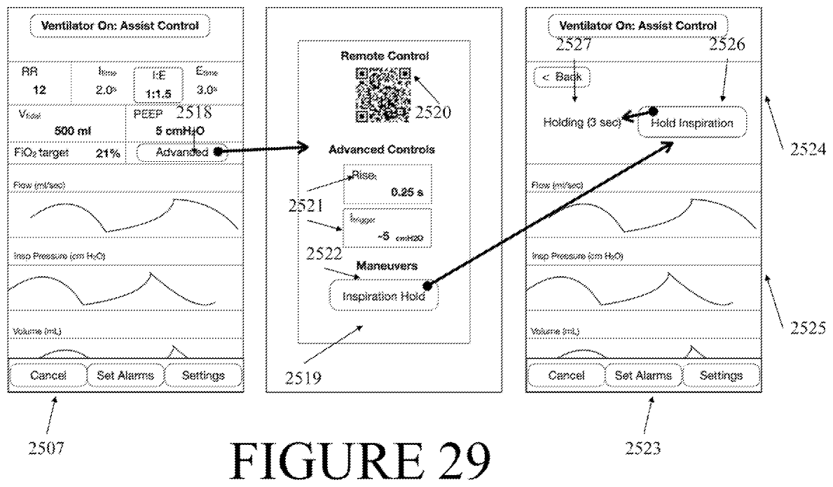

FIG. 29 depicts a series of perspectives, screens, and windows in an advanced menu of a user interface. An operating screen 2507 can include an advanced button 2518. Upon user interaction with an advanced button 2518 an advanced settings window 2519 can be displayed.

An advanced settings window 2519 can present a QR code 2520 to facilitate connecting a patient computer to a remote device (not shown) for telemedicine functionality. An advanced control settings window 2519 can include advanced controls 2521 for settings such as inspiratory rise time or inspiration trigger pressure level. An advanced control settings window 2519 can present options to perform advanced therapeutic and diagnostic techniques, such as an inspiratory hold via an advanced operation button 2522.

Upon user interaction with an advanced operation button 2522 an advanced operating screen 2523 can be displayed with a first advanced delineated section 2524 to display control settings relating to an advanced setting and a second advanced delineated section 2525 to display feedback about the advanced operation.

In inspiratory hold advanced mode, a first advanced delineated section 2524 can display a hold button 2526 and a timer 2527. A medical device (not shown) can effect an inspiratory hold while a hold button is pressed. A timer 2527 can display the time elapsed during the hold.

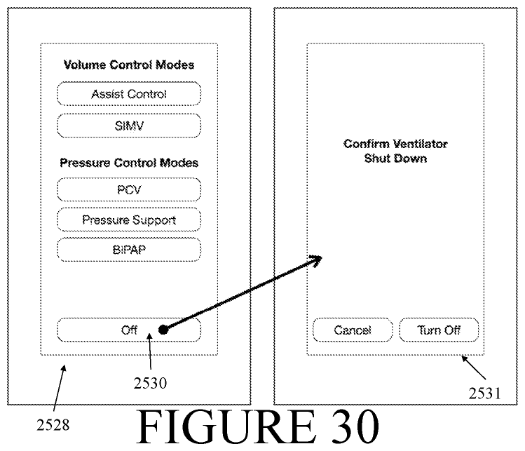

FIG. 30 depicts a series of perspectives, screens, and windows for shutting down a patient device or changing a device mode. After a user interaction with a mode-setting button (not shown) a mode-setting window 2528 can be displayed. After a user interaction with a shut-down button 2530, a shutdown confirmation window 2531 can be displayed A patient computer can return to a home screen (not shown) after receiving a shut-down command.

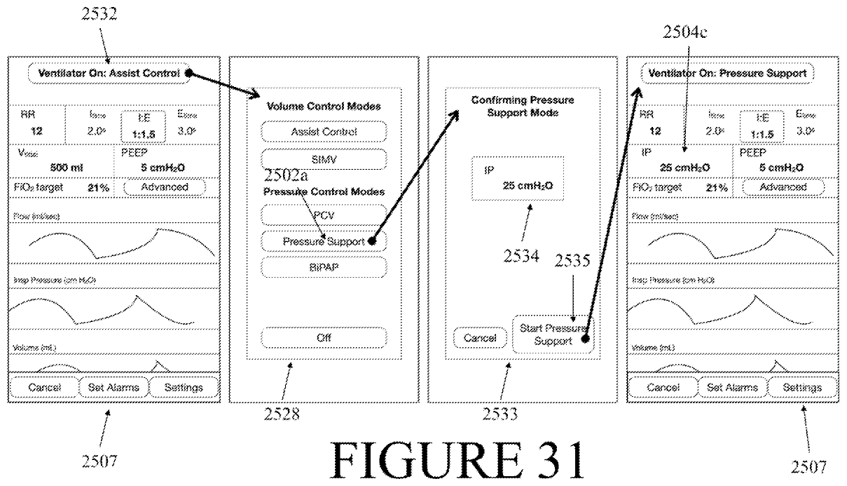

FIG. 31 depicts a series of perspectives, screens, and windows for configuring a pressure support mode. An operating screen 2507 can include a mode-setting button 2532. After a user interaction with a mode-setting button 2532 a mode-setting window 2528 can be displayed. A mode-setting window 2528 can display setting input mechanisms 2534 for settings that are required for the new mode, but not the present mode to avoid changing active settings.

A mode-setting window 2528 can include a pressure support mode button 2502a.

After a user interaction with a pressure support mode button 2502a, a pressure support mode setting window can be displayed 2533. The window 2533 can include a pressure support setting input mechanism 2534 to accept an air pressure level for a desired inspiratory pressure.

After a user action with a confirmation button 2535 an operating screen 2507 can be displayed including an inspiratory pressure control setting 2504c.

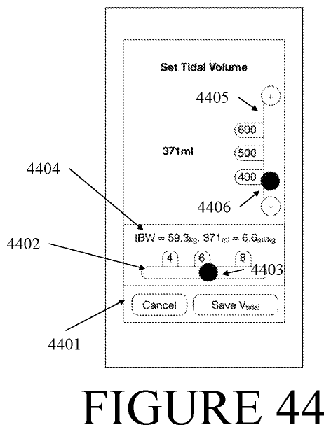

FIG. 44 depicts a series of perspectives, screens, and windows for setting a tidal volume. A tidal-volume setting window 4401 can display a first slider 4402 with a handle 4403. An information display 4404 can display information relevant to the setting, such as a formula for determining a tidal volume for a patient based on ideal body weight (IBW). A first slider 4402 can be used to set a volume per kilogram of IBW, depending on the pathology.

A second slider 4405 can set a total tidal volume responsive to the setting on the first slider 4402. A user can also set a total tidal volume directly using a handle 4406 for a second slider.

A user interface can be used to configure alarms associated with a medical device. A user interface can display a set of configured alarms, each of which can be configured based on, e.g., user-defined criteria and learned events. A display of a configured alarm can indicate a monitored value. A display of a configured alarm can indicate a value that triggers an alarm. A display of a configured alarm can indicate a range that triggers an alarm. A trigger value or range can be determined by a user.

Text can be displayed when an alarm is triggered. A user interface can be used to configure the text to be displayed when an alarm is triggered. Text to be displayed when an alarm is triggered can indicate actions to be taken by a care provider. Text to be displayed when an alarm is triggered can indicate actions to be taken by a care provider in response to a detected condition. Text can display information about treatment history or device operation history, such as measured values over time, historical events, or detected symptoms or conditions. A user can interact with a text display to view additional details, such as a close-up of a graph, a recommended list of actions, or further information about the text already displayed. A device can include a priority list for alarms to display the most relevant alarm if multiple alarms are triggered.

Alarm settings can be customized on a patient-by-patient level. Alarms can be configured to respond to different users and care providers via different communications mechanisms, such as text messages or emails. Alarms can be configured to facilitate responses from remote care providers.

A user interface can be used to infer whether a user is nearby. A computing device associated with a medical device can infer whether a user is nearby based on time elapsed since an interaction with a user interface. A computing device associated with a medical device can infer whether a user is nearby using a motion sensor. A computing device associated with a medical device can infer whether a user is nearby by communicating with a device associated with a user using a ranged communications protocol, such as Bluetooth, near-field communication, or radio frequency. A computing device can identify the nearest provider and determine operations useful to that clinician, such as facilitating authentication, providing preferred alert mechanisms, or pre-populating logging information.

A user interface can utilize a first alarm mechanism if a user is inferred to be nearby. A user interface can utilize a second alarm mechanism if a user is inferred not to be nearby. A second alarm mechanism can be louder than a standard patient alarm. A first alarm can be quieter. A first alarm can display a banner on a user interface.

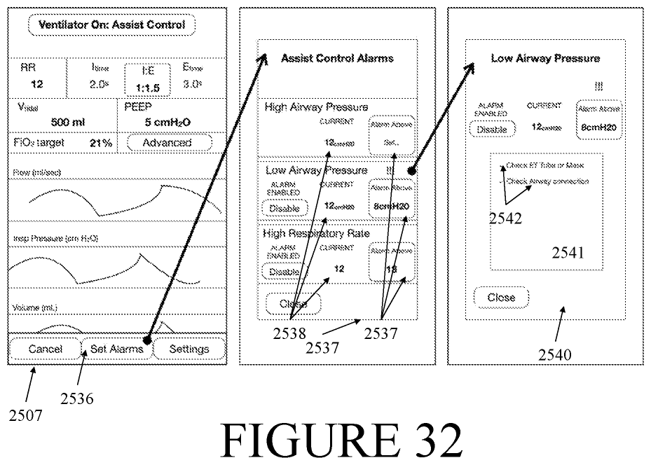

FIG. 32 depicts a series of perspectives, screens, and windows for a user interface for configuring an alarm. An operating screen 2507 can present an alarm-setting button 2536. After a user interaction with an alarm-setting button 2536, an alarm setting window 2537 can be presented. An alarm window can present a list of alarms keyed to various metrics, including threshold levels 2538. Threshold levels 2538 can be presented alongside present levels 2539.

After a user interaction with a threshold level 2538, an alarm-setting window 2540 can be displayed. An alarm-setting window can include a text box 2541 to include instructions 2542 to display on a user interface screen if an alarm is triggered.

A user interface for a medical device can provide audio output capability. A user interface can provide a loud alarm sound. A loud alarm sound can indicate a patient condition. A loud alarm sound can indicate an operating status. A loud alarm sound can indicate a need for therapeutic intervention. Audio effects may indicate different kinds of alarms or required interventions.

A user interface for a medical device can provide text-to-voice capability. A user interface can speak to a user of a medical device. A user interface can speak to a user of a medical device. A user interface can speak instructions to a user of a medical device. A user interface can speak instructions to a user of a medical device responsive to patient conditions. A user interface can speak instructions to a user of a medical device responsive to an operation mode of a medical device.

A user interface for a medical device can provide voice-to-text capability to interact with alarms, such as to silence them, request audio information, change a setting, or confirm an action.

A user interface can provide sound effects for notable events. Notable events can include mode changes, automated mode changes, boot up, or shut down. Notable events can also include changes to patient conditions.

A medical device can include sensors to detect the presence of a person near a medical device. A medical device can include sensors to detect the presence of a medical professional near a medical device.

A sensor can be a motion detector. A sensor can be an infrared sensor. A sensor can be a near-field communication sensor. A sensor can be a radio-frequency fob detector. A sensor can be a Bluetooth connector. A sensor can be a microphone. A sensor can be an array of microphones.

A medical device can detect the presence of touch interactions on a user interface. A medical device can store information about the time elapsed since the last touch interaction on a user interface.

A medical device can modify a user interface responsive to the presence or absence of a person near a medical device.

A medical device can play auditory alarms at a loud volume if no person is near a medical device. A medical device can play auditory alarms at a lower volume if no person is near a medical device.

A user interface for a medical device can provide tutorials relating to use of a medical device. Tutorials can be auditory. Tutorials can be visual. Tutorials can be multi-media. Tutorials can be provided in multiple languages. Tutorials specific to a user's skill level can be provided. Tutorials can be responsive to operation of a medical device. Tutorials can run drills to improve a user's skill level with a medical device.

A simulator of a user interface of a medical device can be provided. A simulator can be provided via a mobile device application. A simulator can be provided via a website application. A simulator of a user interface of a medical device can include tutorials. A simulator can be based on a model of a patient condition.

A medical device with a patient computer can provide telemedicine capabilities.

A user interface for a medical device can provide a web browser. A web browser can be used to facilitate interactions between a patient and a remote provider. A remote provider can be remote to avoid adding contaminants to a patient's room. A remote provider can be remote to avoid contracting contaminants from a patient's room. A remote provider can be remote to provide medical services in a geographic area without adequate providers for a given practice.

Telemedicine functionality can provide monitoring capabilities to a remote provider. Telemedicine functionality can provide data collection and analysis on remote computing devices.

Telemedicine functionality can provide control capabilities to a remote provider.

Telemedicine functionality can facilitate documentation of patient conditions and records. Telemedicine functionality can facilitate provider workflows. Telemedicine functionality can facilitate collaboration among providers. Telemedicine functionality can facilitate collaboration among providers with different specialties. Telemedicine functionality can facilitate collaboration among providers in different geographies.

Telemedicine functionality can facilitate data gathering. Telemedicine functionality can facilitate data gathering in esoteric public health circumstances. Telemedicine functionality can facilitate data ownership schemes and to allow individuals and organizations to receive compensation in exchange for relevant data.

A patient application can be provided. A patient application can receive information from a telemedicine suite. A patient application can transmit information to a telemedicine suite. A patient application can be an application for a mobile device. A patient application can be an application for a website.

A patient application can provide a patient access to information about a patient's health. A patient application can facilitate communication between patients and providers. A patient application can provide information about treatments. A patient application can present patient choices to a patient. A patient application can present patient choices to a patient. A patient application can transmit information to direct payors, such as health insurers.

A patient application can provide functionality relating to inbound patients. A patient application can provide marketing tools. Marketing tools can connect a patient to desired medical products or services. A patient application can provide an onboarding interface for new patients.

A patient application can connect a patient to a medical professional. A patient application can provide for appointment scheduling. A patient application can provide for remote consultation with a medical provider. A patient application can provide for remote consultation by voice. A patient application can provide for remote consultation by video. A patient application can provide for remote consultation by text. A patient application can provide for remote consultation by multimedia mechanism.

A patient application can include a mechanism for data input. A patient application can accept data from at-home measurements, including height, weight, blood pressure, or heartrate. A patient application can interface with smart home health devices. A patient application can accept a patient photograph, video, or audio for identification. A patient application can accept information about a patient lifestyle, including diet and exercise.

A patient application can accept photographs of visible patient conditions. A patient application can accept photographs or scans of healthcare documents, such as insurance cards

A patient application can allow a patient to view the information shared with a provider. A patient application can deliver test results. A patient application can maintain a historical log. A patient application can analyze trends in patient conditions.

A patient application can provide information about a healthcare decision. A patient application can educate a patient or others about risks and tradeoffs of a medical decision. A patient application can provide a verifiable process for informed consent.

A patient application can provide information and decision-making access to approved family, friends, or representatives.

A patient application can provide access to billing information. A patient application can provide access to insurance information.

All of the methods disclosed and claimed herein can be made and executed without undue experimentation in light of the present disclosure. While the apparatus and methods of this invention have been described in terms of preferred embodiments, it will be apparent to those of skill in the art that variations may be applied to the methods and in the steps or in the sequence of steps of the method described herein without departing from the concept, spirit and scope or the invention. In addition, from the foregoing it will be seen that this invention is one well adapted to attain all the ends and objects set forth above, together with other advantages. It will be understood that certain features and sub-combinations are of utility and may be employed without reference to other features and sub-combinations. This is contemplated and within the scope of the invention. All such similar substitutes and modifications apparent to those skilled in the art are deemed to be within the spirit and scope of the invention.

1. A patient interface manifold for modulating air-flow rate comprising:
a casing having a plurality of chambers;
a first peripheral air-flow port connected to a first chamber of the casing;
a second peripheral air-flow port connected to a second chamber of the casing;
a patient air-flow port connected to a third chamber of the casing;
a valve wherein the position of the valve regulates the air-flow rate in the plurality chambers of the casing; and
processing circuitry including computing logic to control the valve to modulate the air-flow rate in the plurality of chambers.

2. The patient interface manifold of claim 1, wherein the valve is a rotary disc valve having a first rotary disc and a second rotary disc, the first rotary disc comprises two apertures, the first rotary disc further substantially fixed to align the apertures of the first rotary disc with each of the chambers of the casing, the second rotary disc comprising one aperture, the second rotary disc configured to rotate.

3. The patient interface manifold of claim 2 wherein the apertures of the second rotary disc have a concave curved edge.

4. The patient interface manifold of claim 1 further comprising a plurality of air-flow port bodies communicatively coupled to the processing circuitry, a first air-flow port body disposed in the first chamber, and a second air-flow port body disposed in the second chamber, the air-flow port bodies configured to detect air-flow rate, the air-flow port bodies further configured to communicate information about a detected air-flow rate to the processing circuitry.

5. The patient interface manifold of claim 4 wherein the plurality of air-flow port bodies have a pitot tube geometry.

6. The patient interface manifold of claim 1 further comprising a sensor communicatively coupled to the processing circuitry wherein the sensor detects one of the temperature, humidity, or gas content of the first chamber.

7. The patient interface manifold of claim 1 wherein the processing circuitry can transmit information to a patient computer.

8. A diagnostic tool comprising:
a face mask having an air-intake port, a first check valve integrated into the air-intake port, an air-exhaust port, and a second check valve integrated into the air-exhaust port;
a casing coupled to the face mask having an air-intake chamber coupled to the air-intake port and an air-exhaust chamber coupled to the air-exhaust port;
a plurality of sensors; and
processing circuitry communicatively coupled to the plurality of sensors, the processing circuitry having computing logic for handling information detected by the plurality of sensors.

9. The diagnostic tool of claim 8, wherein:
the computing logic is capable of directing storage of the information detected by the plurality of sensors.

10. The diagnostic tool of claim 8, wherein the plurality of sensors includes a two-electrode echocardiogram sensor.

11. The diagnostic tool of claim 8, wherein the plurality of sensors includes a skin-temperature sensor.

12. The diagnostic tool of claim 8, wherein the plurality of sensors includes a blood-oxygen sensor.

13. The diagnostic tool of claim 8, wherein the plurality of sensors includes a pulse sensor.

14. The diagnostic tool of claim 8, wherein the plurality of sensors includes a blood-pressure sensor.

15. The diagnostic tool of claim 8, wherein the plurality of sensors includes a first gas-content sensor disposed in the air-intake chamber and a second gas-content sensor disposed in the air-exhaust chamber.

16. The diagnostic tool of claim 8, wherein the plurality of sensors includes a first air-pressure sensor disposed in the air-intake chamber and a second air-pressure sensor disposed in the air-exhaust chamber.

17. A powered air filter comprising:
a casing having an air-flow port couplable to a face mask;
an air blower contained within the casing;
processing circuitry coupled to the air blower, the processing circuitry having computing logic for modulating the speed of the air blower; and
a power source.

18. The powered air filter of claim 17, further comprising:
an air-pressure sensor contained within the casing, the air-pressure sensor communicatively coupled to the processing circuitry, the air-pressure sensor configured to detect an air pressure between the air blower and the air-flow port; and
the processing circuitry is capable of modulating the speed of the air blower based on information detected by the air-pressure sensor.

19. The powered air filter of claim 18, wherein:
the processing circuitry is further capable of modulating the speed of the air blower based on a state-space model; and
wherein the state-space model includes the velocity of the air blower.

20. The powered air filter of claim 17, wherein the casing is constructed from air-filter material.
